(19) United States
(12) Patent Application Publication
(10) Pub. No.: US 2025/0154162 A1
(43) Pub. Date: May 15, 2025

(54) COMPOUND FOR REGULATING AND CONTROLLING 15-PGDH ACTIVITY AND PREPARATION METHOD THEREFOR

(71) Applicant: SCINNOHUB PHARMACEUTICAL CO., LTD., Chengdu, Sichuan (CN)

(72) Inventors: Anle YANG, Chengdu, Sichuan (CN); Sen JI, Chengdu, Sichuan (CN); Hao WANG, Chengdu, Sichuan (CN); Dewei ZHANG, Chengdu, Sichuan (CN); Xiao WANG, Chengdu, Sichuan (CN); Xinying QIAN, Chengdu, Sichuan (CN); Zhiyong LI, Chengdu, Sichuan (CN); Xiaodong ZHANG, Chengdu, Sichuan (CN); Jun TANG, Chengdu, Sichuan (CN)

(21) Appl. No.: 18/833,217

(22) PCT Filed: Jan. 20, 2023
(86) PCT No.: PCT/CN2023/073312

§ 371 Date: Jul. 25, 2024

(30) Foreign Application Priority Data
Jan. 28, 2022 (CN) 202210104578.6

(51) Int. Cl. C07D 495/04; C07D 519/00

(52) U.S. Cl. CPC C07D 495/04; C07D 519/00

(57) ABSTRACT

The present application relates to a compound represented by formula (I) and used for regulating and controlling the 15-PGDH activity and an application thereof in the pharmaceutical field. The present application further provides a method for preparing the compound of the present application, a composition comprising the compound of the present application, and a pharmaceutical use of the compound in serving as a 15-PGDH inhibitor.

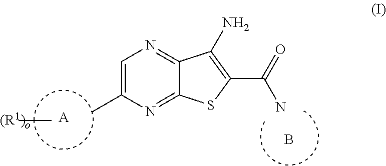

### TECHNICAL FIELD

The present application relates to a compound for regulating 15-PGDH activity and a preparation method thereof, more specifically, relates to a compound useful as a medicament for regulating 15-PGDH activity, and a pharmaceutically acceptable salt thereof, a composition comprising the compound or the salt thereof, and use thereof for preparing a medicament, belonging to the pharmaceutical chemistry field.

### BACKGROUND

15-Hydroxyprostaglandin dehydrogenase (15-PGDH) belongs to an evolutionarily conserved superfamily of short-chain dehydrogenases/reductases (SDRs), and has been designated as SDR³⁶C₁ according to the recently approved nomenclature for human enzymes. Based on current research results, the majority of the in vivo activity can be attributed to HPGD gene-encoded type I 15-PGDH. 15-PGDH plays an important role in the inactivation of active prostaglandins (PGD2, PGE1, PGE2, PGF2α, PGI2, etc.), hydroxyeicosatetraenoic acid (HETE), and inflammation-resolving lipid mediators (RvD1, RvD2, RvE1, MaR¹, LXA4, etc.) (hereinafter referred to generally as 15-PGDH substrates) (e.g., by catalyzing the oxidation reaction of the hydroxyl group at position 15 of PGF2α into 15-keto-PGF2α). These 15-PGDH substrates exert their functions through specific receptors present on target cells. Among others, prostaglandins PGE1, PGE2, PGF2α, PGI2, etc. are often used to assess 15-PGDH activity. For example, PGDH activity is assessed by testing ketone metabolites of the 15-position hydroxyl group of PGF2α (Journal of Clinical Endocrinology and Metabolism, Vol 84, No. 1, 291-299).

Receptors of 15-PGDH substrates are widely and differentially distributed in living organisms, and the diversity of expression distributions, receptor types, and signaling together create a diversity of functions in vivo. For example, PGE1 acts on blood vessels and platelets to promote an increase in bleeding flow by vasodilatory effects and inhibition of platelet aggregation, and is therefore commonly used for treating diseases such as chronic arterial occlusion (thromboangiitis obliterans (TAO) or occlusive arteriosclerosis obliterans (ASO)), skin ulcers; PGF2α has uterine constriction and intraocular pressure lowering effects, and derivatives thereof have been used as therapeutic agents for glaucoma; and PGD2 inhibits inflammation by enhancing the barrier function of the pulmonary blood vessels. In addition, PGE2 has vasodilatory effects, and also a variety of functions, including multiple effects related to blood pressure, pain, bone formation and cell growth, stem cell differentiation, and anti-fibrotic and anti-inflammatory effects, etc. PGI2 has an inhibitory effect on platelet activation and a relaxing effect on vascular smooth muscle, and its derivatives are used as therapeutic agents for chronic arterial occlusion and primary pulmonary hypertension. Inflammation-resolving lipid mediators (RvD1, RvD2, RvE1, MaR¹, LXA4, etc.) inhibit migration/activation of neutrophils and accelerate apoptosis of neutrophils. In addition, it is indispensable in the process of increasing the phagocytic activity of macrophages to effectively remove apoptotic neutrophils/tissue debris remaining at the site of inflammation. These functions promote inflammation and maintain homeostasis within the organism. These inflammation-resolving lipid mediators have been reported to show medicinal efficacy in various types of pathology models (e.g., mouse pulmonary inflammation model, colitis model, and liver injury model).

Recent studies have indicated that 15-PGDH inhibitors and 15-PGDH agonists may have therapeutic value. A recent study showed increased expression of 15-PGDH in the protection against thrombin-mediated cell death. It is well known that 15-PGDH causes inactivation of prostaglandin E2 (PGE2), which is a downstream product of COX-2 metabolism. PGE2 has been shown by research to be beneficial in a variety of biological processes, such as maintaining hair density, promoting skin wound healing, and bone formation.

15-PGDH as an important enzyme in the inactivation of 15-PGDH substrates involves a wide range of in vivo roles, and 15-PGDH inhibitors may be used to prevent or treat diseases associated with 15-PGDH and/or 15-PGDH substrates and/or when there is a need to increase the level of 15-PGDH substrates in a subject.

As mentioned above, some substrates of 15-PGDH have anti-fibrotic, anti-inflammatory, blood flow improvement, growth-promoting, stem cell increase-promoting, smooth muscle contraction/relaxation-promoting, immunosuppression- and bone metabolism-affecting effects, etc. Thus, 15-PGDH inhibitors may effectively treat or prevent fibrosis (e.g., pulmonary fibrosis (idiopathic pulmonary fibrosis, etc.), hepatic fibrosis, renal fibrosis, myocardial fibrosis, scleroderma, and myelofibrosis), inflammatory diseases (e.g., chronic obstructive pulmonary disease (COPD), acute lung injuries, sepsis, deterioration of lung disease and asthma, inflammatory bowel disease (e.g., ulcerative colitis and Crohn's disease), peptic ulcers (e.g., NSAID-induced ulcers), autoinflammatory diseases (e.g., Behçet's disease), vasculitis syndromes, acute liver injury, acute kidney injury, nonalcoholic steatohepatitis (NASH), atopic dermatitis, psoriasis, interstitial cystitis, prostatitis syndrome (e.g., chronic prostatitis/chronic pelvic pain syndrome)), and cardiovascular disease (e.g., pulmonary arterial hypertension, angina pectoris, myocardial infarction, heart failure, ischemic heart disease, chronic kidney disease, renal failure, cerebral apoplexy, and peripheral circulatory disorders), trauma (e.g., diabetic ulcers, burns, pressure ulcers, acute mucosal injuries (including Stevens-Johnson syndrome and mucosal injuries associated with alkylating agents, inhibitors of DNA synthesis, and inhibitors of DNA gyrase, with antimetabolites and other anticancer chemotherapeutic agents, with cellular humoral immunotherapy, or with graft-versus-host disease, such as mucositis or stomatitis)), autoimmune diseases (e.g., multiple sclerosis or rheumatoid arthritis), graft-versus-host disease (GVHD), hair growth, osteoporosis, ear diseases (e.g., hearing loss, tinnitus, vertigo, and dysequilibrium), eye diseases (e.g., glaucoma and dry eye), diabetes mellitus, underactive bladder, neutropenia, neurological diseases caused by transplantation of stem cells, bone marrows or organs (e.g., psychoneurosis, neuropathies, neurotoxic diseases, neuropathic pain, and neurodegenerative diseases), and muscle regenerative diseases (e.g., muscular dystrophy, myodystrophy, and muscle injuries); in addition, 15-PGDH inhibitors may also be used to promote cervical ripening.

The compounds and the pharmaceutically acceptable salts thereof provided in the present application further fulfill the need for small molecules that inhibit 15-PGDH activity.

### SUMMARY OF THE INVENTION

One aspect of the present application provides a compound represented by formula (I), stereoisomers, tautomers or mixed forms thereof, or pharmaceutically acceptable salts thereof, or solvates (e.g., hydrates) thereof, or prodrugs thereof:

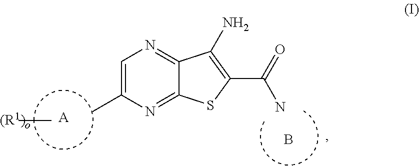

ring A is selected from an aromatic ring, an aromatic heterocycle, an unsaturated aliphatic heterocycle;
ring B is a 5-12 membered saturated aliphatic heterocycle or a ring formed by a 5-12 membered saturated aliphatic heterocycle fused with a benzene ring;
o is selected from 0, 1, 2, 3;
R¹ is each independently selected from deuterium, tritium, nitro, hydroxyl, mercapto, halogen, cyano, ═O, imino, an amine group, an ester group, an aldehyde group, carboxyl, amido, C₃-C₈ cycloalkyl, C₁-C₆ alkyl, C₂-C₆ alkenyl, C₁-C₆ alkoxy, a 3-8 membered saturated aliphatic heterocycle;
or when o is selected from 2, 3, any two R¹ together with atoms of ring A to which they are connected form a 3-8 membered alicyclic group, a 3-8 membered aliphatic heterocyclic group;
wherein the aromatic heterocycle, the saturated aliphatic heterocycle, the unsaturated aliphatic heterocycle, and the aliphatic heterocyclic group each independently comprise 1-3 heteroatoms independently selected from N, O, S, and the ring B comprises at least 1 nitrogen atom;
the ring B, and R¹ are optionally substituted by one or more independently selected from deuterium, tritium, nitro, hydroxyl, —NH₂, mercapto, halogen, cyano, an ester group, carboxyl, amido, ═O, ═NH, C₁-C₆ alkyl, C₁-C₆ alkoxy, C₃-C₈ cycloalkyl, 6-10 membered aryl, 5-10 membered heteroaryl; and furthermore, the ring B is preferably a monocycle, a fused ring or a spirocycle.

In some embodiments, ring A is selected from an aromatic ring, an aromatic heterocycle, an unsaturated aliphatic heterocycle;
ring B is a 5-12 membered saturated aliphatic heterocycle or a ring formed by a 5-12 membered saturated aliphatic heterocycle fused with a benzene ring;
o is selected from 0, 1, 2, 3;
R¹ is each independently selected from deuterium, tritium, nitro, hydroxyl, mercapto, halogen, cyano, ═O, imino, an amine group, an ester group, an aldehyde group, carboxyl, amido, C₃-C₈ cycloalkyl, C₁-C₆ alkyl, C₁-C₆ alkoxy, a 3-8 membered saturated aliphatic heterocycle;
or when o is selected from 2, 3, any two R¹ together with atoms of ring A to which they are connected form a 3-8 membered alicyclic group, a 3-8 membered aliphatic heterocyclic group;
wherein the aromatic heterocycle, the saturated aliphatic heterocycle, the unsaturated aliphatic heterocycle, and the aliphatic heterocyclic group each independently comprise 1-3 heteroatoms independently selected from N, O, S, and the ring B comprises at least 1 nitrogen atom;
the ring B, and R¹ are optionally substituted by one or more independently selected from deuterium, tritium, nitro, hydroxyl, —NH₂, mercapto, halogen, cyano, an ester group, carboxyl, amido, ═O, ═NH, C₁-C₆ alkyl, C₁-C₆ alkoxy, C₃-C₈ cycloalkyl, 6-10 membered aryl, 5-10 membered heteroaryl;
furthermore, the ring B is preferably a monocycle, a fused ring or a spirocycle.

Furthermore, in some embodiments of the present application, the above-mentioned ring B is selected from

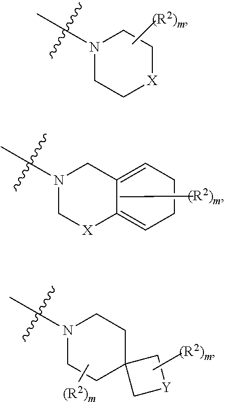

wherein X is selected from a covalent bond, O, S, NH, (CH₂)ₙ, SO₂; Y is selected from a covalent bond, S, NH, (CH₂)ₙ, SO₂;
m is selected from 0, 1, 2, 3; R² is each independently selected from deuterium, tritium, nitro, hydroxyl, mercapto, cyano, halogen, an amine group, an ester group, an aldehyde group, carboxyl, amido, C₁-C₆ alkyl, C₁-C₆ haloalkyl, C₁-C₆ alkoxy, C₃-C₈ cycloalkyl, 6-10 membered aryl, 5-10 membered heteroaryl; and n is selected from 1, 2, 3.

Alternatively, in some embodiments of the present application, the above-mentioned ring B is selected from

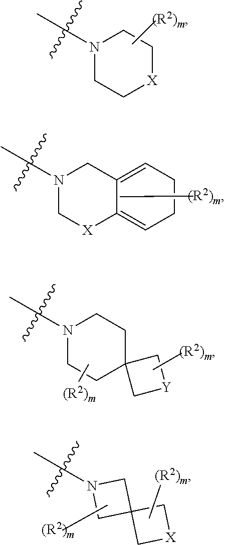

wherein m and R² are as defined above in the present application.

Furthermore, in some specific embodiments, the ring B is selected from

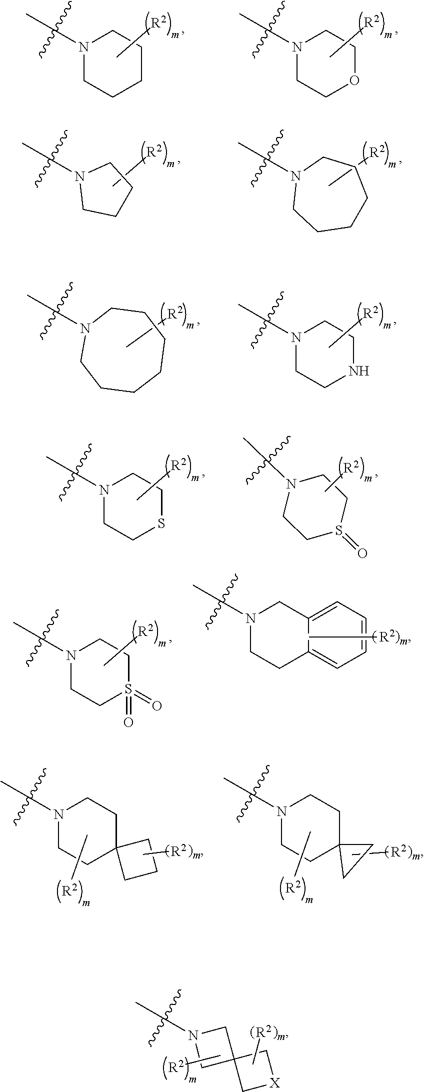

wherein m and R² are as defined above in the present application.

Furthermore, in some specific embodiments, the ring B is selected from

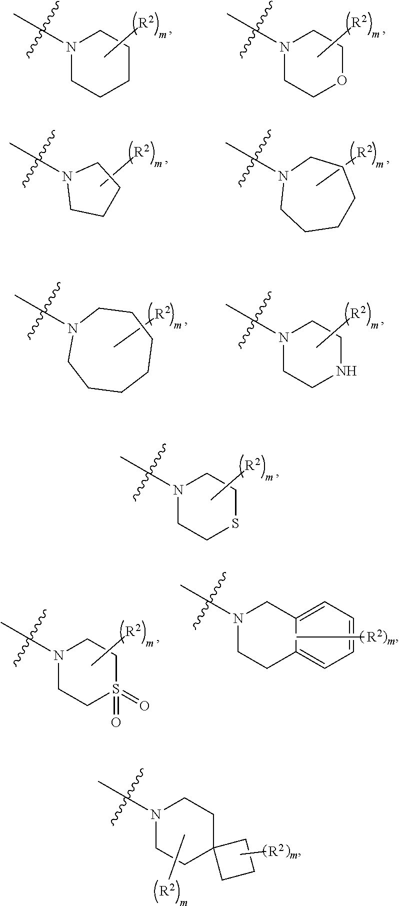

wherein m and R² are as defined above in the present application.

Furthermore, in some specific embodiments of the present application, the above-mentioned R² is each independently selected from deuterium, tritium, nitro, hydroxyl, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, pyridyl.

Furthermore, the ring A is selected from a 6-10 membered aromatic ring, a 5-10 membered aromatic heterocycle, a 6-8 membered unsaturated aliphatic heterocycle; preferably, the aromatic ring and the aromatic heterocycle are a monocycle or a fused ring, the unsaturated aliphatic heterocycle is a monocycle, and the aromatic heterocycle and the unsaturated aliphatic heterocycle each independently comprise 1-3 heteroatoms independently selected from N, O, S.

In an embodiment of the present application, the present application also provides a compound represented by formula (II), stereoisomers, tautomers or mixed forms thereof, or pharmaceutically acceptable salts thereof, or solvates (e.g., hydrates) thereof, or prodrugs thereof:

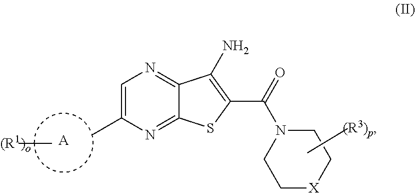

wherein ring A, R¹, and o are as defined above in the present application;
wherein X is selected from a covalent bond, S, NH, CH₂, (CH₂)₂ or (CH₂) 3; R³ is each independently selected from deuterium, tritium, nitro, hydroxyl, mercapto, cyano, halogen, an amine group, an ester group, an aldehyde group, carboxyl, amido, C₁-C₆ alkyl, C₁-C₆ haloalkyl, C₁-C₆ alkoxy, C₃-C₈ cycloalkyl, 6-10 membered aryl, 5-10 membered heteroaryl; p is selected from 0, 1;
preferably, the R³ is each independently selected from deuterium, tritium, nitro, hydroxyl, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, pyridyl.

Furthermore, in an embodiment of the present application, p is 0;

Furthermore, in an embodiment of the present application, p is 0 and X is CH₂.

In an embodiment of the present application, the ring A described in the present application is selected from 6-10 membered aromatic ring, 5-10 membered aromatic heterocycle, 6-8 membered unsaturated aliphatic heterocycle.

Furthermore, the aromatic ring and the aromatic heterocycle are preferably a monocycle or a fused ring, the unsaturated aliphatic heterocycle is preferably a monocycle, and the aromatic heterocycle and the unsaturated aliphatic heterocycle each independently comprise 1-3 heteroatoms independently selected from N, O, S.

In some embodiments of the present application, the ring A is selected from

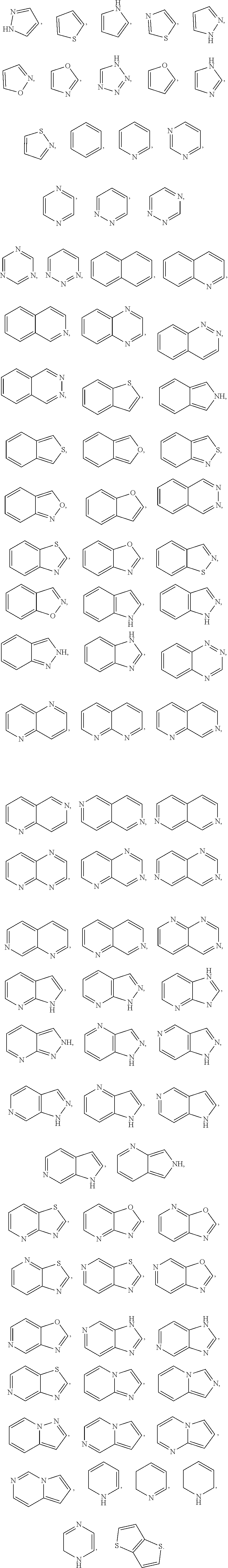

In some specific embodiments of the present application, the ring A is preferably selected from

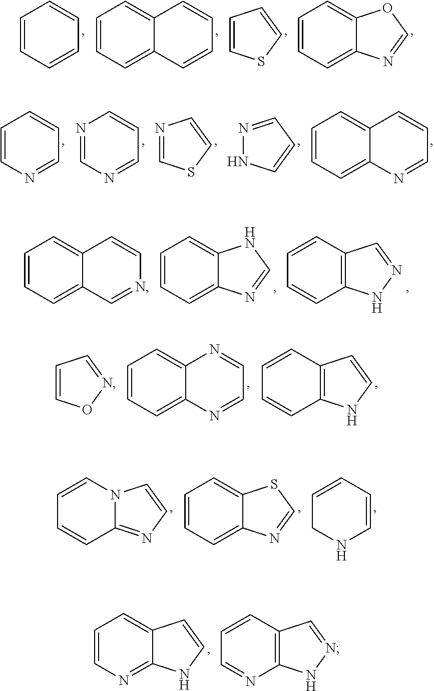

the ring A is more preferably selected from

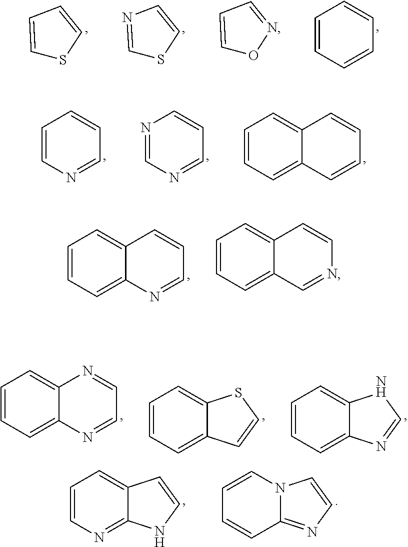

In some embodiments of the present application, R¹ described in the present application is each independently selected from deuterium, tritium, nitro, hydroxyl, mercapto, halogen, cyano, ═O, imino, an amine group, an ester group, an aldehyde group, carboxyl, amido, cyclopropyl, cyclobutyl, cyclohexyl, cyclopentyl, methyl, ethyl, isopropyl, n-butyl, isobutyl, t-butyl, n-pentyl, iso-pentyl, tert-amyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, n-butoxy, isobutoxy, t-butoxy, n-pentoxy, isopentoxy, tert-pentyloxy, n-hexyloxy; or any two R¹ together with atoms of ring A to which they are connected form dioxanyl, dioxolanyl, dioxenyl, dioxolyl, dihydropyridyl, 3-pyrrolinyl, wherein R¹ is optionally substituted by one or more independently selected from deuterium, tritium, nitro, hydroxyl, —NH₂, mercapto, halogen, cyano, an ester group, carboxyl, amido, ═O, ═NH, C₁-C₆ alkyl, C₁-C₆ alkoxy, C₃-C₈ cycloalkyl, 6-10 membered aryl, 5-10 membered heteroaryl;
preferably, R¹ described in the present application is each independently selected from deuterium, tritium, nitro, hydroxyl, mercapto, halogen, cyano, ═O, imino, an amine group, an ester group, an aldehyde group, carboxyl, amido, cyclopropyl, cyclobutyl, cyclohexyl, cyclopentyl, methyl, ethyl, isopropyl, n-butyl, isobutyl, t-butyl, n-pentyl, iso-pentyl, tert-amyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, n-butoxy, isobutoxy, t-butoxy, n-pentoxy, isopentoxy, tert-pentyloxy, n-hexyloxy; or any two R¹ together with atoms of ring A to which they are connected form dioxanyl, dioxolanyl, wherein the R¹ is optionally substituted by one or more independently selected from deuterium, tritium, nitro, hydroxyl, —NH₂, mercapto, halogen, cyano, an ester group, carboxyl, amido, ═O, ═NH, C₁-C₆ alkyl, C₁-C₆ alkoxy, C₃-C₈ cycloalkyl, 6-10 membered aryl, 5-10 membered heteroaryl.

In certain specific embodiments of the present application, R¹ described in the present application is each independently preferably selected from deuterium, tritium, nitro, hydroxyl, mercapto, cyano, ═O, ═NH, —NH₂, —N(CH₃)₂, —NHCH₃, ═NCH₃, an ester group, an aldehyde group, carboxyl, amido, cyclopropyl, cyclobutyl, cyclohexyl, cyclopentyl, methyl, ethyl, isopropyl, trifluoromethyl, trifluoroethyl, trichloromethyl, trichloroethyl, morpholinyl, piperidinyl, N-methylpiperazinyl, p-methylpiperidinyl, piperazinyl, methoxy, ethoxy, isopropoxy, halogen; or any two R¹ together with atoms of ring A to which they are connected form 1,4-dioxanyl, 1,3-dioxanyl, 1,3-dioxolanyl, 1,4-dioxenyl, 1,3-dioxenyl, 1,3-dioxolyl, N-methyl-2-pyridinonyl, N-methyl-3-pyrrolin-2-onyl;
preferably, R¹ described in the present application is each independently preferably selected from deuterium, tritium, nitro, hydroxyl, mercapto, cyano, ═O, ═NH, —NH₂, —N(CH₃)₂, —NHCH₃, ═NCH₃, an ester group, an aldehyde group, carboxyl, amido, cyclopropyl, cyclobutyl, cyclohexyl, cyclopentyl, methyl, ethyl, isopropyl, trifluoromethyl, trifluoroethyl, trichloromethyl, trichloroethyl, morpholinyl, piperidinyl, N-methylpiperazinyl, p-methylpiperidinyl, piperazinyl, methoxy, ethoxy, isopropoxy, halogen; or any two R¹ together with atoms of ring A to which they are connected form 1,4-dioxanyl, 1,3-dioxanyl, 1,3-dioxolanyl.

In certain preferred embodiments, the groups formed by the two R¹ together with atoms of ring A to which they are connected have the following structures: 1,4-dioxanyl having a structure of

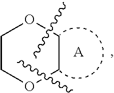

1,3-dioxolanyl having a structure of

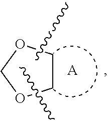

1,4-dioxenyl being able to have a structure of

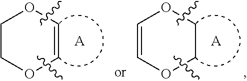

1,3-dioxenyl having a structure of

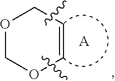

1,3-dioxolyl having a structure of

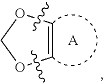

N-methyl-2-pyridinonyl being able to have a structure of

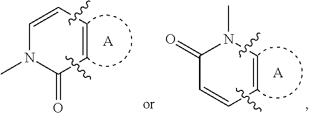

N-methyl-3-pyrrolin-2-onyl having a structure of

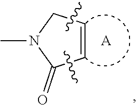

In some embodiments, the present application provides a compound represented by formula (I), stereoisomers or mixed forms thereof, or pharmaceutically acceptable salts thereof, or solvates (e.g., hydrates) thereof, or prodrugs thereof:

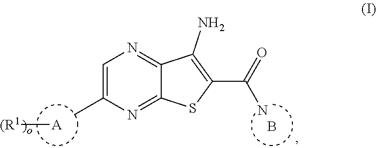

wherein,
ring A is selected from 6-10 membered aromatic ring (e.g., 6 membered aromatic ring, 10 membered aromatic ring), 5-10 membered aromatic heterocycle (e.g., 5 membered aromatic heterocycle, 6 membered aromatic heterocycle, 9 membered aromatic heterocycle, 10 membered aromatic heterocycle), 5-7 membered unsaturated aliphatic heterocycle (preferably 6 membered unsaturated aliphatic heterocycle, e.g., heterocyclohexene or heterocyclohexadiene comprising 1-2 heteroatoms selected from N, O or S);
ring B is a 5-9 membered saturated aliphatic heterocycle (e.g., 5 membered heterocycloalkyl, 6 membered heterocycloalkyl, 7 membered heterocycloalkyl or 8 membered heterocycloalkyl comprising 1-3 heteroatoms selected from N, O or S), or a ring formed by a 5-7 membered saturated aliphatic heterocycle (e.g., 5 membered heterocycloalkyl, 6 membered heterocycloalkyl or 7 membered heterocycloalkyl comprising 1-3 heteroatoms selected from N, O or S) fused with a benzene ring;
o is selected from 0, 1, 2;
R¹ is each independently selected from deuterium, tritium, hydroxyl, halogen, cyano, ═O, imino, an amine group, amido, C₅-C₇ cycloalkyl, C₁-C₆ alkyl, C₂-C₆ alkenyl, C₁-C₆ alkoxy, 5-7 membered saturated aliphatic heterocycle; or any two R¹ together with atoms of ring A to which they are connected form a 5-6 membered alicyclic group, a 5-6 membered aliphatic heterocyclic group;
wherein the aromatic heterocycle, the saturated aliphatic heterocycle, the unsaturated aliphatic heterocycle, and the aliphatic heterocyclic group each independently comprise 1-3 heteroatoms independently selected from N, O, S, and the ring B comprises at least 1 nitrogen atom;
the ring B is a monocycle, a fused ring or a spirocycle, and is optionally substituted by one or more independently selected from deuterium, tritium, hydroxyl, halogen (e.g., fluoro, chloro, bromo), ═O, C₁-C₆ alkyl, C₁-C₆ alkoxy, C₃-C₅ cycloalkyl (e.g., one or more selected from halogen, ═O, C₁-C₆ alkyl);
the R¹ is optionally substituted by one or more independently selected from deuterium, tritium, C₁-C₆ alkyl, C₁-C₆ alkoxy, C₃-C₆ cycloalkyl.

In some embodiments, ring A is selected from benzene ring, naphthalene, thiophene, benzoxazole, pyridine, pyrimidine, thiazole, pyrazole, pyrrole, imidazole, quinoline, isoquinoline, benzimidazole, indazole, pyrazolopyridine, oxazole, isoxazole, quinoxaline, indole, imidazopyridine, benzothiazole, pyrrolopyridine, azacyclohexadiene.

In some preferred embodiments, ring A is selected from benzene ring, naphthalene, thiophene, benzoxazole, pyridine, pyrimidine, thiazole, pyrazole, quinoline, isoquinoline, benzimidazole, indazole, pyrazolopyridine, isoxazole, quinoxaline, indole, imidazopyridine, benzothiazole, pyrrolopyridine, azacyclohexadiene.

In some embodiments, ring B is piperidine, halopiperidine or dihalopiperidine (e.g., fluoropiperidine, difluoropiperidine, chloropiperidine, dichloropiperidine, bromopiperidine, dibromopiperidine, etc.), C₁-C₆ alkylpiperidine or di(C₁-C₆ alkyl) piperidine (e.g., C₁-C₅ alkylpiperidine, di(C₁-C₅ alkyl) piperidine, C₁-C₄ alkylpiperidine, di(C₁-C₄ alkyl) piperidine, C₁-C₃ alkylpiperidine, di(C₁-C₃ alkyl) piperidine, methylpiperidine, dimethylpiperidine, ethylpiperidine, diethylpiperidine, propylpiperidine, dipropylpiperidine), hexamethyleneimine, thiomorpholine, morpholine, C₁-C₆ alkylmorpholine or di(C₁-C₆ alkyl) morpholine (e.g., C₁-C₅ alkylmorpholine, di(C₁-C₅ alkyl) morpholine, C₁-C₄ alkylmorpholine, di(C₁-C₄ alkyl) morpholine, C₁-C₃ alkylmorpholine, di(C₁-C₃ alkyl) morpholine, methylmorpholine, dimethylmorpholine, ethylmorpholine, diethylmorpholine, propylmorpholine, dipropylmorpholine), pyrrolidine, halopyrrolidine or dihalopyrrolidine (e.g., fluoropyrrolidine, difluoropyrrolidine, chloropyrrolidine, dichloropyrrolidine, bromopyrrolidine, dibromopyrrolidine), piperazine, C₁-C₆ alkylpiperazine or di(C₁-C₆ alkyl) piperazine (e.g., C₁-C₅ alkylpiperazine, C₁-C₄ alkylpiperazine, C₁-C₃ alkylpiperazine, N-methylpiperazine, N-ethylpiperazine, N-propylpiperazine), thiomorpholine-1,1-dioxide, thiomorpholine-1-oxide, tetrahydroisoquinoline, azaspirooctane, oxaazaspiroheptane. In some embodiments, ring B is e.g., piperidine, halopiperidine or dihalopiperidine (e.g., fluoropiperidin, difluoropiperidine, chloropiperidine, dichloropiperidine, bromopiperidine, dibromopiperidine, etc.), C₁-C₆ alkylpiperidine or di(C₁-C₆ alkyl) piperidine (e.g., C₁-C₅ alkylpiperidine, di(C₁-C₅ alkyl) piperidine, C₁-C₄ alkylpiperidine, di(C₁-C₄ alkyl) piperidine, C₁-C₃ alkylpiperidine, di(C₁-C₃ alkyl) piperidine, methylpiperidine, dimethylpiperidine, ethylpiperidine, diethylpiperidine, propylpiperidine, dipropylpiperidine), hexamethyleneimine.

In some embodiments, o is selected from 0, 1, 2;
R¹ is each independently selected from deuterium, tritium, halogen (e.g., fluoro, chloro, bromo), cyano, ═O, imino, an amine group, C₁-C₆ alkyl (e.g., C₁-C₈ alkyl, C₁-C₄ alkyl, C₁-C₃ alkyl, methyl, ethyl, propyl), C₂-C₆ alkenyl (e.g., C₂-C₅ alkenyl, C₂-C₄ alkenyl, C₂-C₃ alkenyl, methyl, vinyl, 1-propenyl, 2-propenyl, isopropenyl), C₁-C₆ alkoxy (e.g., C₁-C₅ alkoxy, C₁-C₄ alkoxy, C₁-C₃ alkoxy, methoxy, ethoxy, propoxy), 6 membered saturated aliphatic heterocycle (e.g., morpholine, dioxane, piperazine, thiomorpholine), amido; or two R¹ together with atoms of ring A to which they are connected form a 5-6 membered aliphatic heterocyclic group comprising 1-2 heteroatoms selected from N or O (e.g., dioxacyclohexenyl (dioxenyl), dioxolyl, dihydropyrrolyl, dihydropyridyl);
R¹ is optionally substituted by one or more independently selected from deuterium, tritium, C₁-C₆ alkyl (e.g., C₁-C₅ alkyl, C₁-C₄ alkyl, C₁-C₃ alkyl, methyl, ethyl, propyl).

In some embodiments, o is selected from 0, 1, 2;
R¹ is each independently selected from deuterium, tritium, fluoro, chloro, bromo, cyano, ═O, imino, an amine group, methyl, ethyl, propyl, methoxy, ethoxy, propoxy, 6 membered saturated aliphatic heterocycle (e.g., morpholine, piperazine); or two R¹ together with atoms of ring A to which they are connected form a 5-6 membered aliphatic heterocyclic group comprising 1-2 heteroatoms selected from N or O (e.g., dioxenyl, dioxolyl); the 6 membered saturated aliphatic heterocyclic group, and the 5-6 membered aliphatic heterocyclic group are optionally substituted by one or more independently selected from deuterium, tritium, C₁-C₆ alkyl (e.g., C₁-C₅ alkyl, C₁-C₄ alkyl, C₁-C₃ alkyl, methyl, ethyl, propyl).

The present application also relates to any combination of the above-mentioned various embodiments or some features therein.

In some specific embodiments of the present application, the present application provides the compounds as shown below, stereoisomers thereof, tautomers or mixed forms thereof, or pharmaceutically acceptable salts thereof, or solvates thereof, or prodrugs thereof:

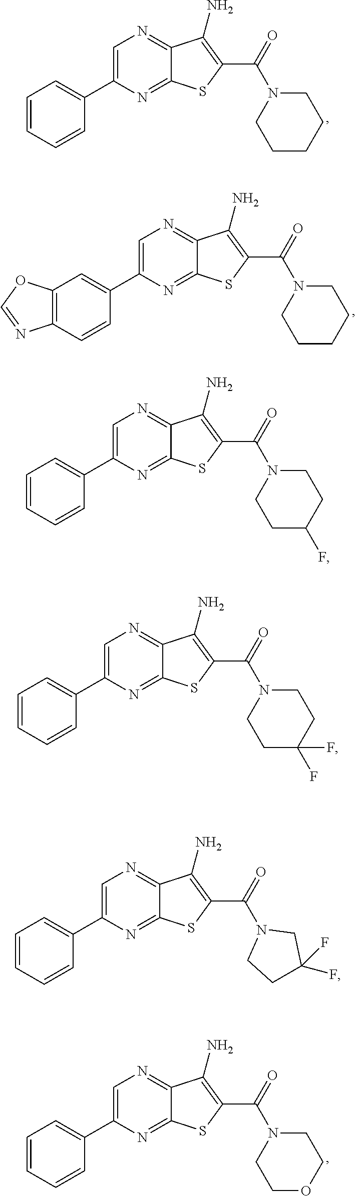 [SMILES: Nc1c(C(=O)N2CCCCC2)sc2nc(-c3ccccc3)cnc12 ; Nc1c(C(=O)N2CCCCC2)sc2nc(-c3ccc4ncoc4c3)cnc12 ; Nc1c(C(=O)N2CCC(F)CC2)sc2nc(-c3ccccc3)cnc12 ; Nc1c(C(=O)N2CCC(F)(F)CC2)sc2nc(-c3ccccc3)cnc12 ; Nc1c(C(=O)N2CCC(F)(F)C2)sc2nc(-c3ccccc3)cnc12 ; Nc1c(C(=O)N2CCOCC2)sc2nc(-c3ccccc3)cnc12]

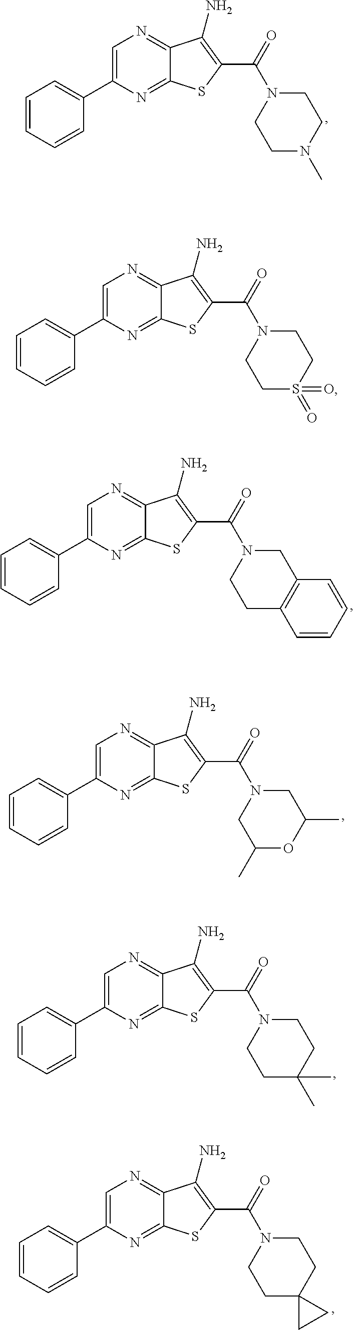 [SMILES: CN1CCN(C(=O)c2sc3nc(-c4ccccc4)cnc3c2N)CC1 ; Nc1c(C(=O)N2CCS(=O)(=O)CC2)sc2nc(-c3ccccc3)cnc12 ; Nc1c(C(=O)N2CCc3ccccc3C2)sc2nc(-c3ccccc3)cnc12 ; CC1CN(C(=O)c2sc3nc(-c4ccccc4)cnc3c2N)CC(C)O1 ; CC1(C)CCN(C(=O)c2sc3nc(-c4ccccc4)cnc3c2N)CC1 ; Nc1c(C(=O)N2CCC3(CC2)CC3)sc2nc(-c3ccccc3)cnc12]

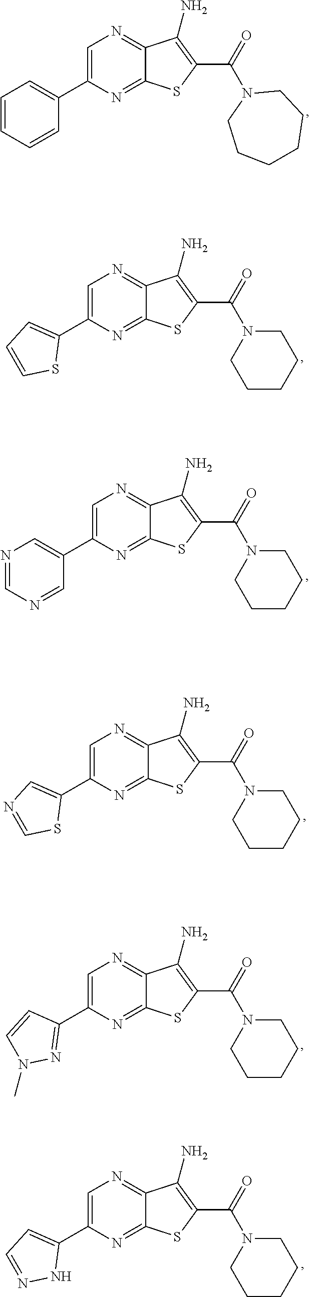 [SMILES: Nc1c(C(=O)N2CCCCCC2)sc2nc(-c3ccccc3)cnc12 ; Nc1c(C(=O)N2CCCCC2)sc2nc(-c3cccs3)cnc12 ; Nc1c(C(=O)N2CCCCC2)sc2nc(-c3cncnc3)cnc12 ; Nc1c(C(=O)N2CCCCC2)sc2nc(-c3cncs3)cnc12 ; Cn1ccc(-c2cnc3c(N)c(C(=O)N4CCCCC4)sc3n2)n1 ; Nc1c(C(=O)N2CCCCC2)sc2nc(-c3ccn[nH]3)cnc12]

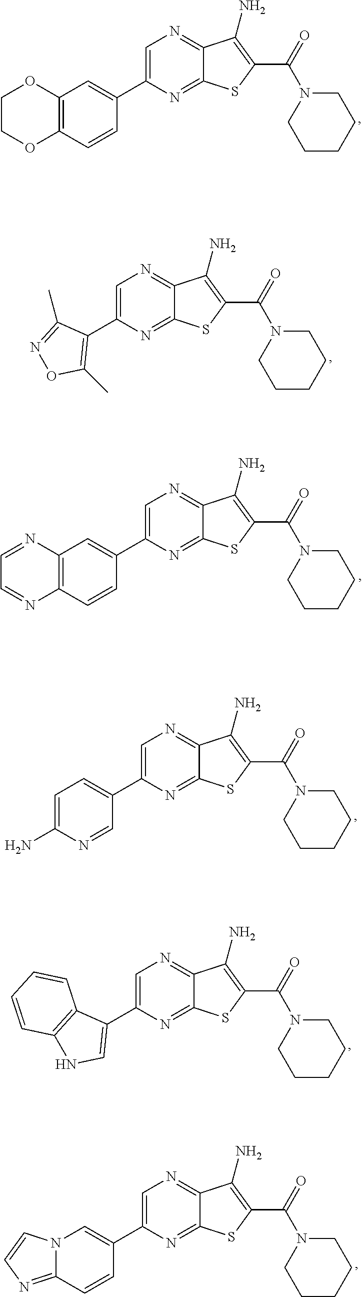 [SMILES: Nc1c(C(=O)N2CCCCC2)sc2nc(-c3ccc4c(c3)OCCO4)cnc12 ; Cc1noc(C)c1-c1cnc2c(N)c(C(=O)N3CCCCC3)sc2n1 ; Nc1c(C(=O)N2CCCCC2)sc2nc(-c3ccc4nccnc4c3)cnc12 ; Nc1ccc(-c2cnc3c(N)c(C(=O)N4CCCCC4)sc3n2)cn1 ; Nc1c(C(=O)N2CCCCC2)sc2nc(-c3c[nH]c4ccccc34)cnc12 ; Nc1c(C(=O)N2CCCCC2)sc2nc(-c3ccc4nccn4c3)cnc12]

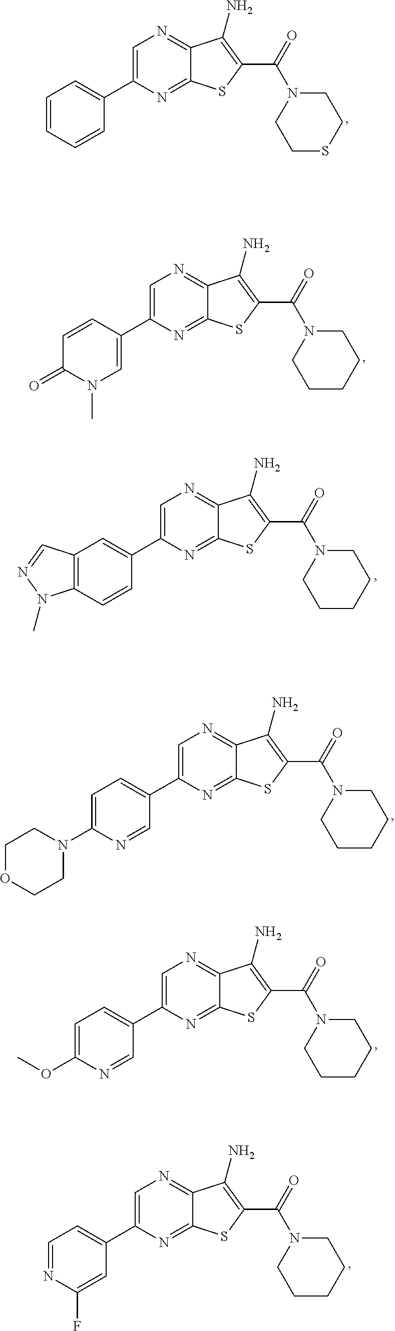 [SMILES: Nc1c(C(=O)N2CCSCC2)sc2nc(-c3ccccc3)cnc12 ; Cn1cc(-c2cnc3c(N)c(C(=O)N4CCCCC4)sc3n2)ccc1=O ; Cn1ncc2cc(-c3cnc4c(N)c(C(=O)N5CCCCC5)sc4n3)ccc21 ; Nc1c(C(=O)N2CCCCC2)sc2nc(-c3ccc(N4CCOCC4)nc3)cnc12 ; COc1ccc(-c2cnc3c(N)c(C(=O)N4CCCCC4)sc3n2)cn1 ; Nc1c(C(=O)N2CCCCC2)sc2nc(-c3ccnc(F)c3)cnc12]

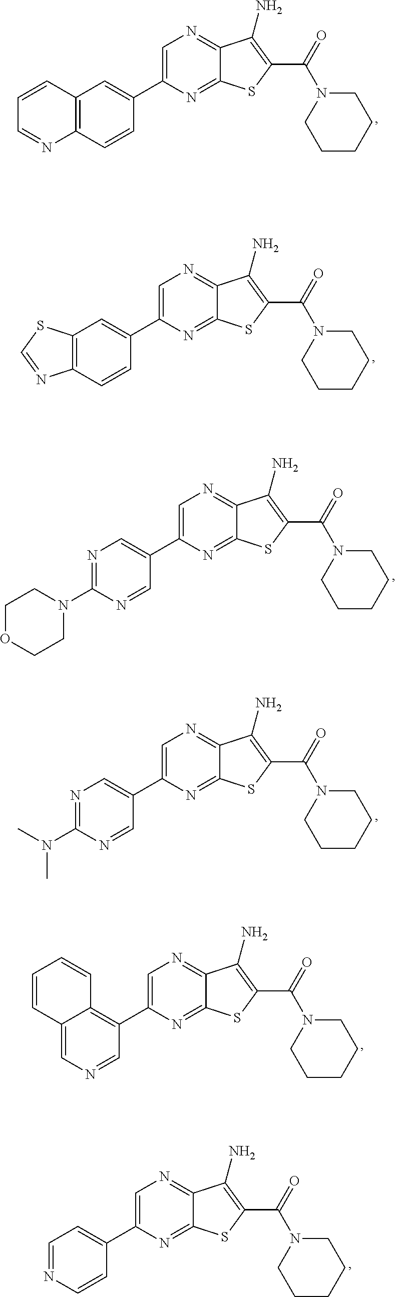 [SMILES: Nc1c(C(=O)N2CCCCC2)sc2nc(-c3ccc4ncccc4c3)cnc12 ; Nc1c(C(=O)N2CCCCC2)sc2nc(-c3ccc4ncsc4c3)cnc12 ; Nc1c(C(=O)N2CCCCC2)sc2nc(-c3cnc(N4CCOCC4)nc3)cnc12 ; CN(C)c1ncc(-c2cnc3c(N)c(C(=O)N4CCCCC4)sc3n2)cn1 ; Nc1c(C(=O)N2CCCCC2)sc2nc(-c3cncc4ccccc34)cnc12 ; Nc1c(C(=O)N2CCCCC2)sc2nc(-c3ccncc3)cnc12]

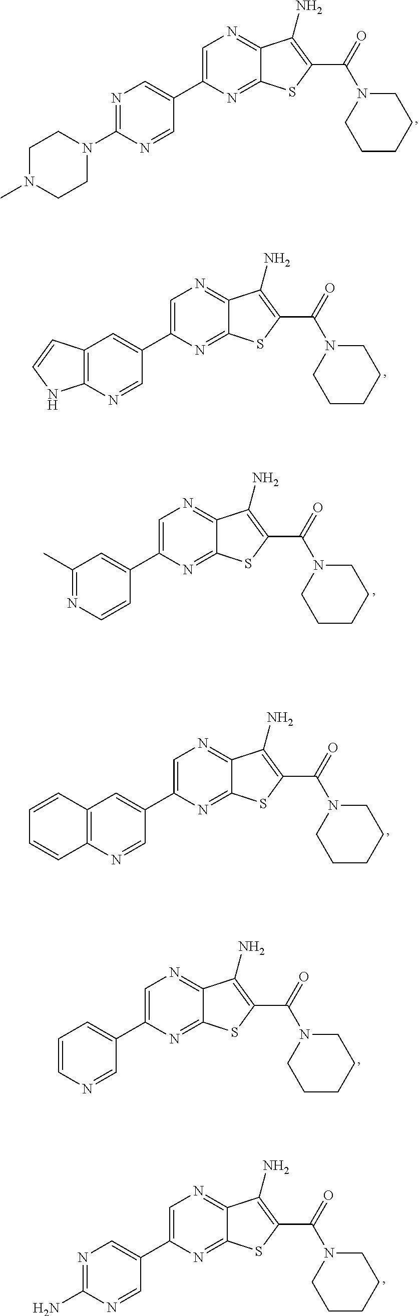 [SMILES: CN1CCN(c2ncc(-c3cnc4c(N)c(C(=O)N5CCCCC5)sc4n3)cn2)CC1 ; Nc1c(C(=O)N2CCCCC2)sc2nc(-c3cnc4[nH]ccc4c3)cnc12 ; Cc1cc(-c2cnc3c(N)c(C(=O)N4CCCCC4)sc3n2)ccn1 ; Nc1c(C(=O)N2CCCCC2)sc2nc(-c3cnc4ccccc4c3)cnc12 ; Nc1c(C(=O)N2CCCCC2)sc2nc(-c3cccnc3)cnc12 ; Nc1ncc(-c2cnc3c(N)c(C(=O)N4CCCCC4)sc3n2)cn1]

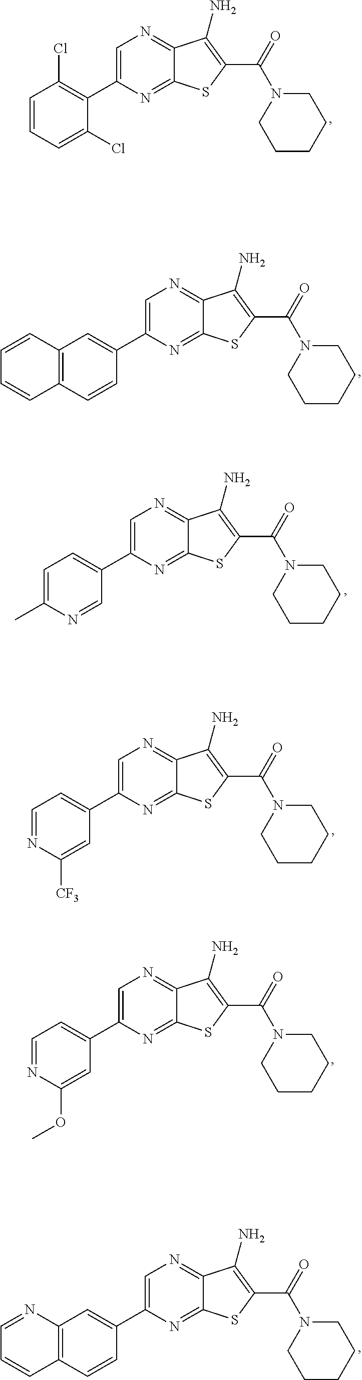 [SMILES: Nc1c(C(=O)N2CCCCC2)sc2nc(-c3c(Cl)cccc3Cl)cnc12 ; Nc1c(C(=O)N2CCCCC2)sc2nc(-c3ccc4ccccc4c3)cnc12 ; Cc1ccc(-c2cnc3c(N)c(C(=O)N4CCCCC4)sc3n2)cn1 ; Nc1c(C(=O)N2CCCCC2)sc2nc(-c3ccnc(C(F)(F)F)c3)cnc12 ; COc1cc(-c2cnc3c(N)c(C(=O)N4CCCCC4)sc3n2)ccn1 ; Nc1c(C(=O)N2CCCCC2)sc2nc(-c3ccc4cccnc4c3)cnc12]

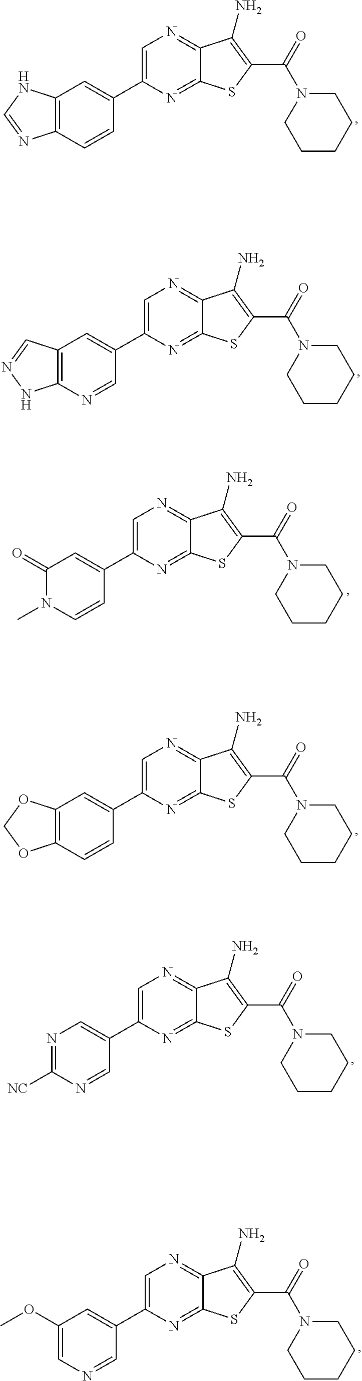 [SMILES: Nc1c(C(=O)N2CCCCC2)sc2nc(-c3ccc4nc[nH]c4c3)cnc12 ; Nc1c(C(=O)N2CCCCC2)sc2nc(-c3cnc4[nH]ncc4c3)cnc12 ; Cn1ccc(-c2cnc3c(N)c(C(=O)N4CCCCC4)sc3n2)cc1=O ; Nc1c(C(=O)N2CCCCC2)sc2nc(-c3ccc4c(c3)OCO4)cnc12 ; N#Cc1ncc(-c2cnc3c(N)c(C(=O)N4CCCCC4)sc3n2)cn1 ; COc1cncc(-c2cnc3c(N)c(C(=O)N4CCCCC4)sc3n2)c1]

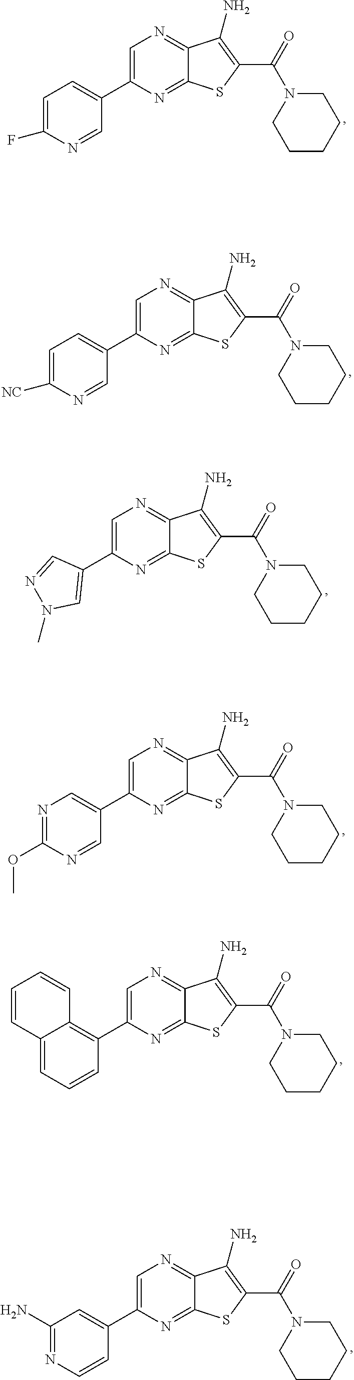 [SMILES: Nc1c(C(=O)N2CCCCC2)sc2nc(-c3ccc(F)nc3)cnc12 ; N#Cc1ccc(-c2cnc3c(N)c(C(=O)N4CCCCC4)sc3n2)cn1 ; Cn1cc(-c2cnc3c(N)c(C(=O)N4CCCCC4)sc3n2)cn1 ; COc1ncc(-c2cnc3c(N)c(C(=O)N4CCCCC4)sc3n2)cn1 ; Nc1c(C(=O)N2CCCCC2)sc2nc(-c3cccc4ccccc34)cnc12 ; Nc1cc(-c2cnc3c(N)c(C(=O)N4CCCCC4)sc3n2)ccn1]

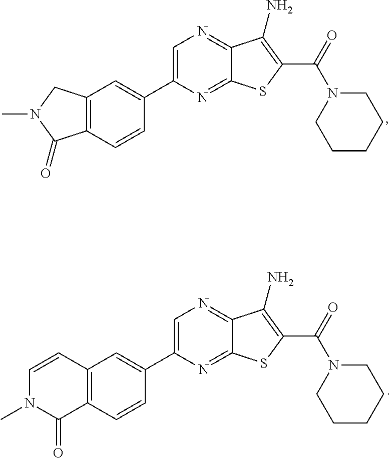 [SMILES: CN1Cc2cc(-c3cnc4c(N)c(C(=O)N5CCCCC5)sc4n3)ccc2C1=O ; Cn1ccc2cc(-c3cnc4c(N)c(C(=O)N5CCCCC5)sc4n3)ccc2c1=O]

Another aspect of the present application provides a pharmaceutical composition comprising at least one of the above-mentioned compounds, stereoisomers, tautomers or mixed forms thereof, or pharmaceutically acceptable salts thereof, or solvates thereof, or prodrugs thereof, and at least one pharmaceutically acceptable excipient.

Another aspect of the present application provides use of the above-mentioned compound, stereoisomers, tautomers or mixed forms thereof, or pharmaceutically acceptable salts thereof, or solvates thereof, or prodrugs thereof, or the pharmaceutical composition for preparing a medicament. Wherein the medicament is a 15-PGDH inhibitor useful for treating a disease associated with the undesirably increased level of 15-PGDH activity. Alternatively, the present application provides the above-mentioned compounds, stereoisomers, or tautomers or mixed forms thereof, or pharmaceutically acceptable salts thereof, or solvates thereof, or prodrugs thereof, or the pharmaceutical composition used as a medicament. Alternatively, the present application provides a method of treating or preventing a 15-PGDH associated disease, comprising administering to a subject in need thereof the above-mentioned compound, stereoisomers, or tautomers or mixed forms thereof, or pharmaceutically acceptable salts thereof, or solvates thereof, or prodrugs thereof, or the pharmaceutical composition. The 15-PGDH associated disease herein refers to a disease or complication thereof for which a clinically beneficial effect such as remission, amelioration, cessation of progression, alleviation, or no further deterioration is achieved by inhibiting 15-PGDH activity.

In certain specific embodiments, the medicament, the inhibitor or the method is useful for treating or preventing fibrosis, oral ulcer, gum disease, colitis, ulcerative colitis, gastroduodenal ulcer, inflammatory disease, vascular insufficiency, Raynaud's disease, Buerger's disease, neuropathy, pulmonary arterial hypertension, cardiovascular and renal disease, cardiovascular disease, trauma, skin damage, autoimmune disease, graft-versus-host disease, osteoporosis, ear disease, eye disease, neutropenia, diabetes mellitus, underactive bladder, or for promoting hair growth, pigmentation, tissue repair, tissue regeneration, implant in stem cell transplantation or bone marrow transplantation or organ transplantation, neurogenesis and neuronal cell death, muscle regeneration and cervical ripening, or for enhancing resistance to the toxicity of radiation exposure, the toxicity of chemotherapy, the toxicity of immunosuppressant.

### Definition

Unless otherwise stated, the following terms used in the specification and claims have the following meanings. A particular term or phrase shall not be considered uncertain or unclear in the absence of a specific definition, but should be understood according to its ordinary meaning.

“Alkyl” refers to a saturated aliphatic hydrocarbon group. The alkyl moiety may be a linear or branched alkyl; C₁-C₆ alkyl used herein refers to a linear or branched alkyl comprising 1 to 6 (e.g., 1, 2, 3, 4, 5, 6, or a range value composed of any two of the preceding numerical values) carbon atoms. Typical alkyl includes, but is not limited to, methyl, ethyl, n-propyl, isopropyl, n-butyl, isobutyl, t-butyl, n-pentyl, isopentyl, tert-amyl, n-hexyl, etc.

“Alkoxy” refers to —O-alkyl; C₁-C₆ alkoxy used herein refers to a linear or branched alkoxy group comprising 1 to 6 (e.g., 1, 2, 3, 4, 5, 6, or a range value composed of any two of the preceding numerical values) carbon atoms. Typical alkoxy includes, but is not limited to, methoxy, ethoxy, n-propoxy, isopropoxy, n-butoxy, isobutoxy, t-butoxy, n-pentyloxy, isopentyloxy, tert-pentyloxy, n-hexyloxy, etc.

“Alkenyl” refers to an aliphatic chain hydrocarbon group comprising a carbon-carbon double bond. The alkenyl moiety may be a linear or branched alkenyl; C₂-C₆ alkenyl used in the present application refers to a linear or branched alkenyl comprising 2 to 6 (e.g., 2, 3, 4, 5, 6, or a range value composed of any two of the preceding numerical values) carbon atoms. Typical alkenyl includes, but is not limited to, vinyl, 1-propenyl, 2-propenyl, isopropenyl, 1-butenyl, 2-butenyl, 3-butenyl, etc.

“Ring” refers to any ring-like covalently closed structure, including, for example, a carbocycle (e.g., an aromatic or alicyclic ring), or a heterocycle (e.g., an aromatic or aliphatic heterocycle). The carbocycle refers to a ring only consisted of carbon atoms, and the heterocycle refers to a closed structure formed by covalently bonding carbon atoms and heteroatoms. The ring may be monocyclic, bicyclic, tricyclic or polycyclic. When the ring is a bicyclic, tricyclic or polycyclic ring, the relationship between individual rings may include a fused ring, a spirocycle, or a bridged cycle. For example, a bicyclic ring may include a spirocycle, a fused ring, and a bridged cycle, and a tricyclic ring may include a trispirocycle, a fused tricycle, a spirocycle fused monocycle, etc.

The term “fused” in the present application refers to the situation of sharing two neighboring ring atoms between rings, e.g., a fused ring refers to a ring-like structure formed by sharing two neighboring atoms between two monocycles.

“Heteroatom” refers to any atom, other than a carbon atom, that can be covalently bonded to a carbon atom. Common heteroatoms include, but are not limited to, O, S, N, P, Si, etc.

The “membered” refers to the number of skeleton atoms constituting a ring. A typical 5-membered ring may include, but is not limited to, cyclopentane, pyrrole, imidazole, thiazole, furan, thiophene, and the like, and a typical 6-membered ring includes, but is not limited to, cyclohexane, pyridine, pyran, pyrazine, thiapyran, pyridazine, pyrimidines, benzene, etc.

“Alicyclic ring” or “alicyclic group” refers to a saturated or partially unsaturated carbocycle; the saturated carbocycle may be referred to as, e.g., a saturated alicyclic ring; the partially unsaturated carbocycle may be referred to as, e.g., an unsaturated alicyclic ring; an alicyclic ring may be consisted of 3-10 atoms, and may be a monocycle or a polycycle; for example, the C₃-C₈ alicyclic group used in the present application refers to an alicyclic group consisted of 3-8 skeleton atoms. A typical alicyclic structure includes, but is not limited to:

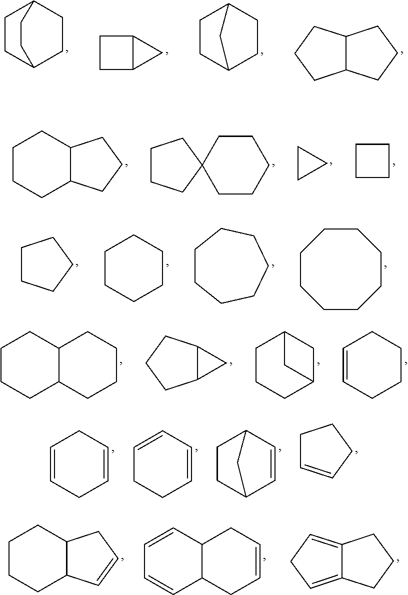

etc.

“Aliphatic heterocycle” or “aliphatic heterocyclic group” refers to a nonaromatic ring-like group formed by replacing carbon atom(s) in an alicyclic ring with one or more heteroatoms. The aliphatic heterocycle or aliphatic heterocyclic group may include a saturated aliphatic heterocycle and an unsaturated aliphatic heterocycle. For example, a 3-8 membered aliphatic heterocyclic group used in the present application refers to a nonaromatic ring-like group comprising one or more heteroatoms consisted of 3-8 skeleton atoms, and may be a saturated aliphatic heterocyclic group and an unsaturated aliphatic heterocyclic group.

“Saturated aliphatic heterocycle” or “saturated aliphatic heterocyclic group” means that the carbon atoms in the aliphatic heterocycle that form the ring skeleton are all saturated. For example, a 5-12 membered saturated aliphatic heterocycle used in the present application refers to a nonaromatic ring-like group formed by 5-12 atoms constituting the ring skeleton, wherein the atoms constituting the ring skeleton comprise saturated carbon atoms and heteroatoms. A typical saturated aliphatic heterocycle includes, but is not limited to:

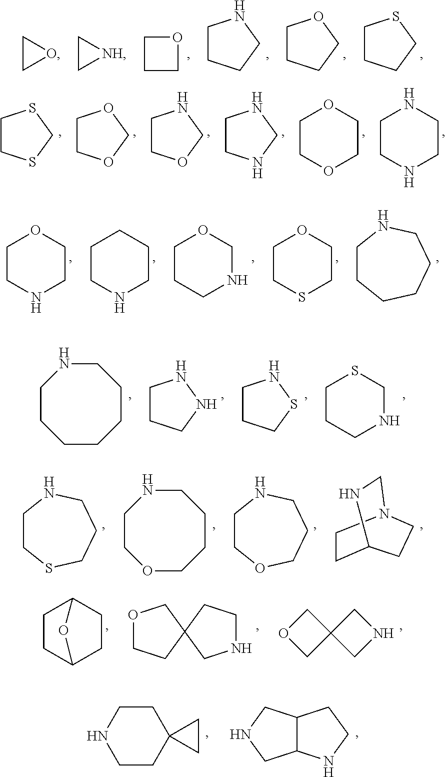

etc.

As used in the application, “a ring formed by a 5-12 membered saturated aliphatic heterocycle fused with a benzene ring” refers to a ring-like structure consisted of a saturated aliphatic heterocycle of 5-12 atoms and a benzene ring in a fused means. For example, etc.

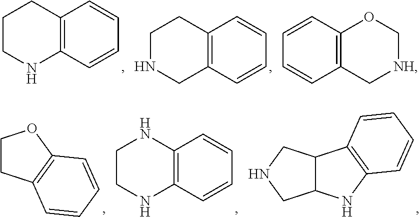

The “unsaturated aliphatic heterocycle” in the application means that the skeleton of the aliphatic heterocycle contains unsaturated carbon atoms. A 6-8 membered unsaturated aliphatic heterocycle used in the present application refers to a nonaromatic ring-like group formed by 6-8 skeleton atoms, wherein the atoms constituting the ring skeleton include saturated carbon atoms, unsaturated carbon atoms, and heteroatoms, and a typical unsaturated aliphatic heterocycle includes, but is not limited to:

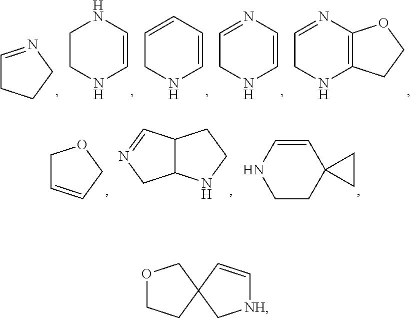

etc.

“Cycloalkyl” refers to a saturated aliphatic carbocyclic group, and may also be referred to as, for example, a saturated alicyclic ring. The cycloalkyl group may be a monocycle, a spirocycle, a fused ring or a bridged cycle. A C₃-C₈ cycloalkyl used in the application refers to a cyclic alkyl comprising 3-8 carbon atoms. A typical cycloalkyl includes, but is not limited to, cyclopropyl, cyclobutyl, cyclopentyl, cyclohexyl, bicyclo[2,1,1]hexyl, cycloheptyl, and the like.

“Aromatic ring” or “aryl” refers to a completely unsaturated carbocycle with a planar ring having a delocalized π-electron system and containing 4n+2π electrons, where n is an integer. The aromatic ring may consist of six, eight, ten, or more than ten carbon atoms, and may be monocyclic or polycyclic. Common aromatic ring includes, but is not limited to, benzene ring, naphthalene ring, phenanthrene ring, anthracene ring, tetrabenzene, pyrene ring, pentabenzene, and the like. As used in the present application, a 6-10 membered aromatic ring or a 6-10 membered aryl group refers to an aromatic ring group consisting of 6-10 skeleton carbon atoms.

“Aromatic heterocycle” or “heteroaryl” refers to an aromatic ring-like structure formed by replacing carbon atoms in the aromatic ring with one or more heteroatoms, and a typical aromatic heterocycle or heteroaryl includes, but is not limited to:

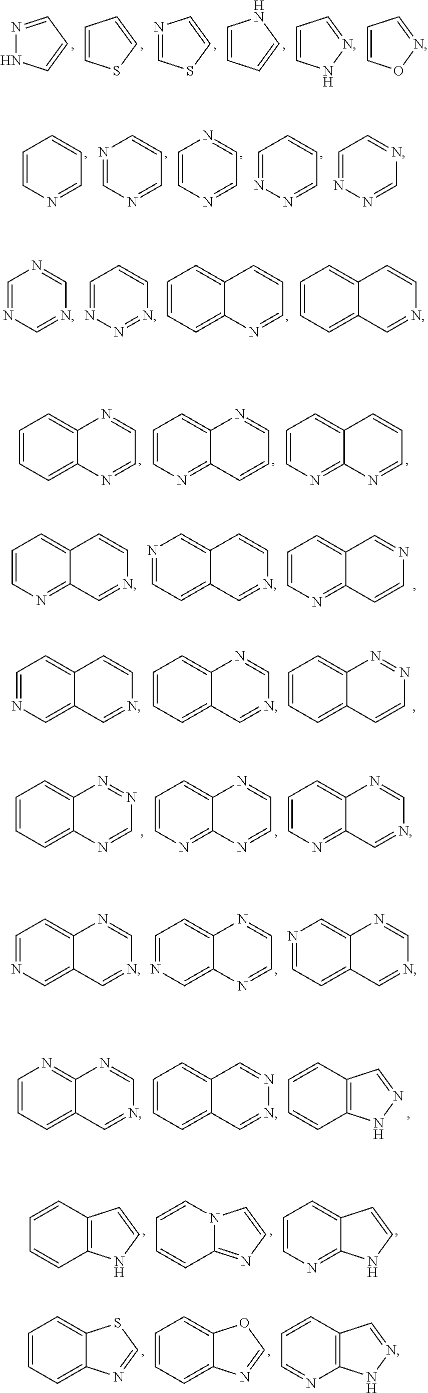

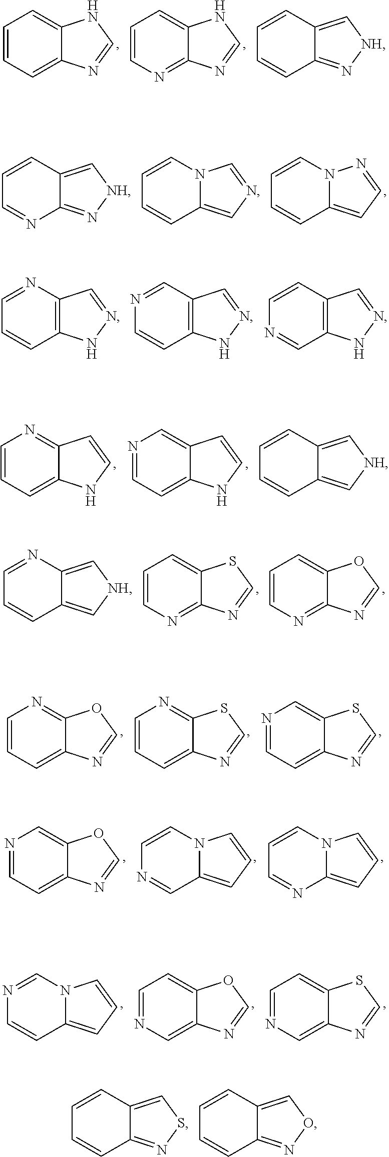

etc.

As used in the present application, a 5-10 membered aromatic heterocycle or a 5-10 membered heteroaryl refers to an aromatic ring group comprising heteroatom(s) consisted of 5-10 skeleton carbon atoms.

The “halogen” or “halo” refers to fluorine, chlorine, bromine or iodine.

“Haloalkyl” means that at least one hydrogen in an alkyl group is replaced by a halogen atom, and a C₁-C₆ haloalkyl, as used in this application, means a linear or branched alkyl consisting of 1-6 carbon atoms and at least one hydrogen in the alkyl is arbitrarily replaced by a halogen atom.

“Amine group” or “amine” means having a chemical structure of —NR^{U}R^{V}, wherein R^{U}, R^{V} are each independently selected from hydrogen, deuterium, tritium, alkyl, cycloalkyl.

“Imino” or “imine” means having a chemical structure of ═NR^{W}, wherein R^{W} is selected from hydrogen, deuterium, tritium, alkyl, cycloalkyl.

“Amide” or “amido” means having a chemical structure of —C(O)NR^{X}R^{Y} or —NR^{X}C(O)R^{Y}, wherein R^{X}, R^{Y} are each independently selected from hydrogen, deuterium, tritium, alkyl, cycloalkyl, and common amido includes, but is not limited to —CONH₂, —CONHCH₃, —CON(CH₃)₂, —NHCOH, —NHCOCH₃, —N(CH₃) COCH₃.

“Ester group” means having a chemical structure of a formula of —COOR⁰, wherein R⁰ is selected from alkyl, cycloalkyl, heterocycloalkyl, aryl, heteroaryl.

“Substituted” means that one or more hydrogen atoms in a group are substituted independently by a corresponding number of substituents. It goes without saying that, the substituents are only in their possible chemical positions, and those of skills in the art are able to determine (either experimentally or theoretically) possible or impossible substitutions without undue effort. For example, an amino or hydroxyl with a free hydrogen may be unstable when binds to a carbon atom with an unsaturated (e.g. olefinic) bond. Each is independently selected from alkyl, cycloalkyl, aryl, heteroaryl, heterocycloalkyl, hydroxy, alkoxy, alkylthio, aryloxy, nitro, acyl, halogen, haloalkyl, amino, and the like. When two or more “substitutions” occur, the substituents may form a cyclic group together with the substituted atoms. For example, in the present application, two R¹ together with the atoms of ring A to which they are connected form a 1,4-dioxanyl group with a structure of

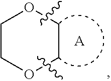

1,3-dioxolanyl with a structure of

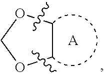

1,4-dioxenyl being able to have a structure of

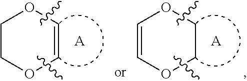

1,3-dioxenyl with a structure of

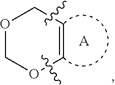

1,3-dioxolyl with a structure of

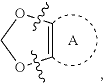

N-methyl-2-pyridinonyl being able to have a structure of

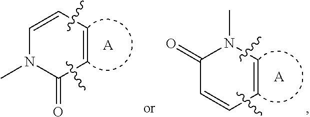

N-methyl-3-pyrrolin-2-onyl with a structure of

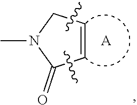

“Inhibitor” refers to a substance reducing the activity of an enzyme.

“Optional” or “optionally” means that the event or circumstance subsequently described may, but not necessarily, occur, and the description includes a situation when the event or circumstance does or does not occur. For example, “optionally substituted” includes substituted or unsubstituted, e.g., “a heterocyclic group optionally substituted by an alkyl” means that the alkyl may, but not necessarily, be present, and the description includes a situation in which the heterocyclic group is substituted by the alkyl and a situation in which the heterocyclic group is not substituted by the alkyl.

“Pharmaceutical composition” indicates a mixture comprising one or more of the compounds described herein, or a physiologically/pharmaceutically acceptable salt or prodrug thereof, with other chemical components, as well as other components such as physiologically/pharmaceutically acceptable carriers and vehicles. The pharmaceutical composition is intended to facilitate administration to an organism and facilitate absorption of an active ingredient to exert the biological activity.

“Pharmaceutically acceptable” refers to those compounds, materials, compositions and/or dosage forms that are suitable for use in contact with human and animal tissues within a range of sound medical judgment, without undue toxicity, irritation, allergic response or other problems or complications, commensurate with a reasonable benefit/risk ratio.

As pharmaceutically acceptable salts, mention may be made of, e.g., metal salts, ammonium salts, salts formed with organic bases, salts formed with inorganic acids, salts formed with organic acids, salts formed with basic or acidic amino acids, and the like.

“Tautomer” or “tautomeric form” refers to structural isomers of different energies that can be interconverted through low-energy barriers. For example, proton tautomer (also known as proton transfer tautomer) includes tautomerism via proton migration, such as keto-enol and imine-enamine isomerization. Specific example of proton tautomer is imidazole moiety, wherein the proton can migrate between the two ring nitrogens. Valence tautomers include interconversion by recombination of some bonding electrons. Non-limiting examples of tautomers include, but are not limited to

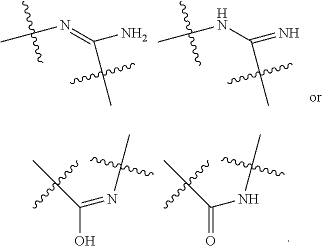 [SMILES: */N=C(\*)N ; *NC(*)=N ; */N=C(\*)O ; *NC(*)=O]

“Stereoisomer” refers to an isomer resulting from a different spatial arrangement of atoms in a molecule.

“Enantiomer” refers to isomerism caused by different spatial configurations of the atoms of compounds having the same molecular formula and functional groups, and said compounds form stereoisomers that are mirror images of each other and cannot overlap.

“Diastereoisomer” refers to isomerism caused by different spatial configurations of the atoms of compounds having the same molecular formula and functional groups, and said compounds are stereoisomers that do not exhibit a physical or mirror image relationship with each other.

Unless otherwise indicated, the terms “comprise, comprises and comprising” or their equivalents (contain, contains, containing, include, includes, including) used herein are open-ended mode expressions, and mean that other unspecified elements, components and steps may also be covered, in addition to the elements, components and steps listed.

Unless otherwise indicated, all numbers used herein to denote amounts of ingredients, measurements, or reaction conditions should be understood to be modified in all cases by the term “about”. When associated with a percentage, the term “about” may indicate, for example, +1%, preferably +0.5%, more preferably +0.1%.

Unless otherwise specified clearly in the context, singular terms herein cover plural referents, and vice versa. Similarly, unless otherwise specified clearly in the context, the word “or” herein is intended to include “and”.

Apparently, according to the above contents of the application, in accordance with the ordinary technical knowledge and means in the field, under the premise of not departing from the above basic technical concepts of the application, a variety of other forms of modifications, substitutions or changes can also be made.

The abbreviations in the application have the meanings indicated below:

| | |
|---|---|
| rt represents a reaction temperature at room temperature; | DMF represents N,N-dimethylformamide; |
| Pd(dppf)Cl₂ represents | THF represents tetrahydrofuran; |
| [1,1′-bis(diphenylphosphino)ferrocene]dichloropalladium; | |
| MeOH represents methanol; | KOH represents potassium hydroxide; |
| HATU represents | DIPEA or DIEA represents |
| 2-(7-azabenzotriazol-1-yl)-N,N,N′,N′-tetramethyluronium | N,N-diisopropylethylamine; |
| hexafluorophosphate; | |
| K₂CO₃ represents potassium carbonate; | Na₂CO₃ represents sodium carbonate; |
| o/n represents reaction overnight; | MeONa represents sodium methoxide; |
| BBr₃ represents a solution of boron tribromide in | DCM represents dichloromethane; |
| tetrahydrofuran; | |
| PhN(Tf)2 represents; | Cs₂CO₃ represents cesium carbonate; |
| DCE represents 1,2-dichloroethane. | |

### Embodiments of the Invention

The methods of synthesizing the compounds and intermediates of the present application are described below by way of example. The following examples are only intended to serve as examples of the present application, and should not be taken as a limitation to the scope of the present application. Unless otherwise indicated, the raw materials and reagents involved in the present application are all available commercially, and the specific source does not affect the implementation of the technical solution of the present application.

### Example 1: Preparation of (7-amino-3-phenylthieno[2,3-b]pyrazin-6-yl) (piperidin-1-yl) methanone

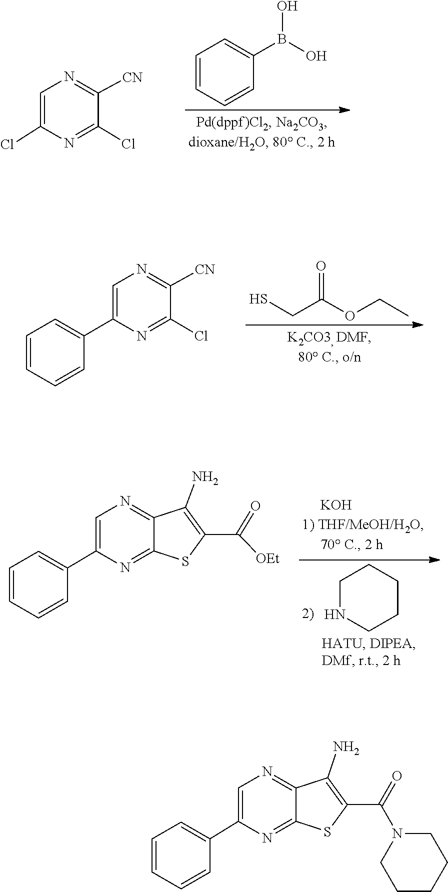

### Step 1: preparation of 3-chloro-5-phenylpyrazine-2-carbonitrile

3,5-dichloropyrazine-2-carbonitrile (2.5 g), phenylboronic acid (1.95 g), sodium carbonate (1.84 g), [1,1′-bis(diphenylphosphino) ferrocene]dichloropalladium (0.53 g) were weighed and dissolved in a mixed solvent of 1,4-dioxane (20 mL) and water (5 mL), reacted at 80° C. for 2 h after nitrogen replacement for three times, and the reaction of raw materials was monitored by TLC until complete. It was cooled to room temperature, filtered. The filtrate was added with water, and extracted with ethyl acetate, and organic phases were combined, dried over anhydrous sodium sulfate, and evaporated under reduced pressure to remove the solvent, and the residue was purified by silica gel column to obtain the crude title compound (3.1 g).

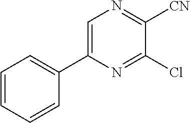 [SMILES: N#Cc1ncc(-c2ccccc2)nc1Cl]

MS (ESI) m/z (M+H)⁺=216.0.

### Step 2: preparation of ethyl 7-amino-3-phenylthieno[2,3-b]pyrazine-6-carboxylate

3-chloro-5-phenylpyrazine-2-carbonitrile (80 mg) was weighed, and dissolved in N,N-dimethylformamide (2 mL), into which potassium carbonate (120 mg), ethyl thioglycolate (54 μL) were added, which were reacted at 80° C. overnight, and the reaction was monitored by LC-MS until complete. It was cooled to room temperature, quenched with water, and extracted with ethyl acetate three times, and the organic phases were combined, dried over anhydrous sodium sulfate, concentrated under reduced pressure, and purified on silica gel column to obtain the title compound (90 mg).

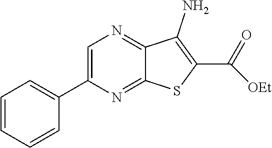 [SMILES: CCOC(=O)c1sc2nc(-c3ccccc3)cnc2c1N]

MS (ESI) m/z (M+H)⁺=300.1.

### Step 3: preparation of (7-amino-3-phenylthieno[2,3-b]pyrazin-6-yl) (piperidin-1-yl) methanone

(1) Ethyl 7-amino-3-phenylthieno[2,3-b]pyrazine-6-carboxylate (90 mg) was weighed, and dissolved in tetrahydrofuran (2 mL), methanol (0.6 mL) and water (0.6 mL), into which potassium hydroxide (51 mg) was added, which were reacted at 70° C. for 2 h, and the reaction was monitored by LCMS until complete. It was cooled to room temperature, added with water, and extracted with ethyl acetate three times. The organic phase was discarded. The aqueous phase was adjusted to pH 2 with 2 M diluted HCl, and then extracted with ethyl acetate, and the organic phases were combined, dried over anhydrous sodium sulfate, and evaporated under reduced pressure to remove the solvent, thereby obtaining 7-amino-3-phenylthieno[2,3-b]pyrazine-6-carboxylic acid crude product.

(2) The crude product obtained above was dissolved in N,N-dimethylformamide (2 mL), and diisopropylethylamine (100 μL), piperidine (37 μL), 2-(7-azabenzotriazol-1-yl)-N,N,N′,N′-tetramethyluronium hexafluorophosphate (170 mg) were sequentially added thereto in ice-water bath, and placed and stirred at room temperature for 2 h. The reaction was monitored by LCMS until complete. The reaction was quenched by addition of water, and it was extracted with ethyl acetate three times, and the organic phases were combined, dried over anhydrous sodium sulfate, and concentrated under reduced pressure, and the residue was purified by reverse phase preparative column to obtain the title compound (9.5 mg).

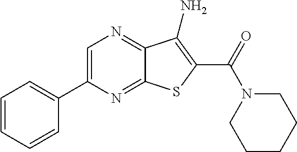 [SMILES: Nc1c(C(=O)N2CCCCC2)sc2nc(-c3ccccc3)cnc12]

MS (ESI) m/z (M+H)⁺=339.1,

1H NMR (400 MHZ, DMSO-d₆) δ 9.34 (s, 1H), 8.25-8.23 (m, 2H), 7.60-7.54 (m, 3H), 6.20 (s, 2H), 3.59-3.57 (m, 4H), 1.64-1.57 (m, 6H).

### Example 2: preparation of (7-amino-3-(benzo[d]oxazol-6-yl) thieno[2,3-b]pyrazin-6-yl) (piperidin-1-yl) methanone

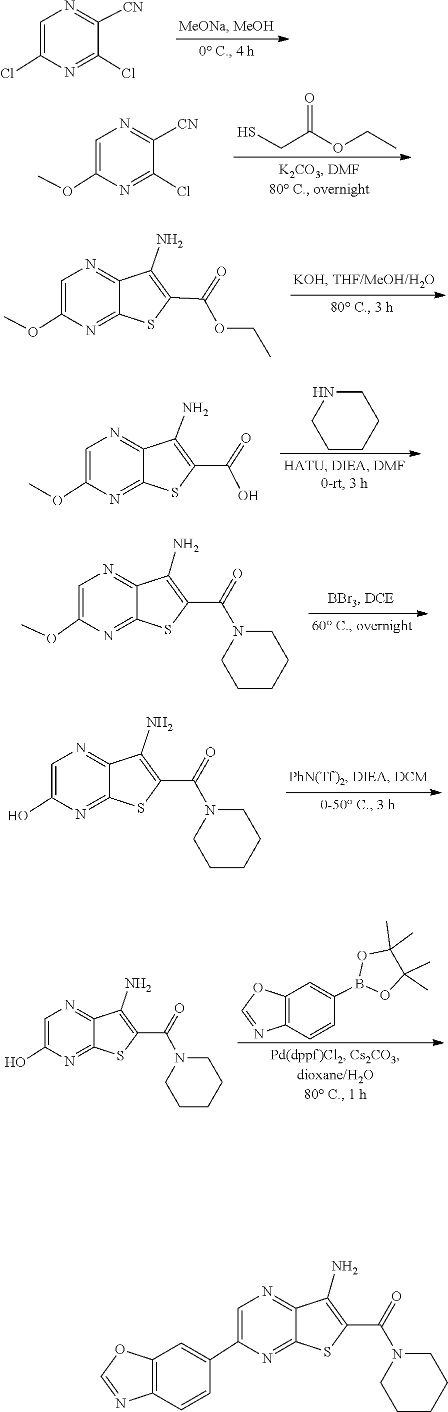

### Step 1: preparation of 3-chloro-5-methoxypyrazine-2-carbonitrile

3,5-dichloropyrazine-2-carbonitrile (8.0 g) was weighed and dissolved in methanol (50 mL), and sodium methoxide (2.5 g) was added thereto at 0° C., which were reacted at 0° C. for 3 h, and thereafter was raised to room temperature with stirring for 1 h. TLC showed that the raw materials were consumed completely, and it was concentrated under reduced pressure, quenched by adding water, extracted twice with ethyl acetate, dried over anhydrous sodium sulfate, and purified by silica gel column chromatography to obtain the title compound (4.317 g).

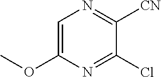

MS (ESI) m/z (M+H)⁺=170.0.

### Step 2: preparation of ethyl 7-amino-3-methoxythieno[2,3-b]pyrazine-6-carboxylate

3-chloro-5-methoxypyrazine-2-carbonitrile (4.317 g) was weighed and dissolved in N,N-dimethylformamide (50 mL), and potassium carbonate (7.76 g), ethyl thioglycolate (3.35 mL) were added thereto, which were reacted at 80° C. overnight. TLC showed that the raw materials were consumed completely, and it was quenched by addition of water, and extracted with ethyl acetate 2 times, washed with saturated brine 2 times, dried over anhydrous sodium sulfate, and concentrated under reduced pressure to obtain the title compound (4.5 g).

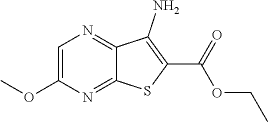 [SMILES: CCOC(=O)c1sc2nc(OC)cnc2c1N]

MS (ESI) m/z (M+H)⁺=254.0.

### Step 3: preparation of 7-amino-3-methoxythieno[2,3-b]pyrazine-6-carboxylic acid

Ethyl 7-amino-3-methoxythieno[2,3-b]pyrazine-6-carboxylate (4.5 g) was weighed and dissolved in a mixed solvent of tetrahydrofuran (24 mL), methanol (8 mL), and water (8 mL), and potassium hydroxide (2.99 g) was added thereto, which were reacted at 80° C. for 3 h. LCMS showed that the raw materials were consumed completely, and it was quenched by adding water, adjusted to an acidic pH value with 2 M hydrochloric acid, extracted with ethyl acetate 3 times, dried over anhydrous sodium sulfate and concentrated under reduced pressure to give the title compound (4.2 g).

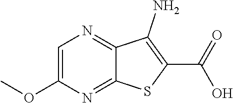

MS (ESI) m/z (M+H)⁺=226.0.

### Step 4: preparation of (7-amino-3-methoxythieno[2,3-b]pyrazin-6-yl) (piperidin-1-yl) methanone

7-amino-3-methoxythieno[2,3-b]pyrazine-6-carboxylic acid (4.2 g) was weighed and dissolved in N,N-dimethylformamide (40 mL), and N,N-diisopropylethylamine (6.18 mL), hexahydropyridine (1.28 mL), 2-(7-azabenzotriazol-1-yl)-N,N,N′,N′-tetramethyluronium hexafluorophosphate (5.32 g) were sequentially added thereto at 0° C., and then it was raised to room temperature to react for 3 h. TLC showed that the raw materials were consumed completely, and it was quenched by addition of water, extracted with ethyl acetate 2 times, washed with saturated brine 2 times, dried over anhydrous sodium sulfate, and purified by silica gel column chromatography to obtain the title compound (2.1 g).

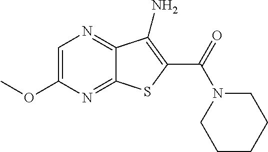 [SMILES: COc1cnc2c(N)c(C(=O)N3CCCCC3)sc2n1]

MS (ESI) m/z (M+H)⁺=293.1.

### Step 5: preparation of (7-amino-3-hydroxylthieno[2,3-b]pyrazin-6-yl) (piperidin-1-yl) methanone

(7-amino-3-methoxythieno[2,3-b]pyrazin-6-yl) (piperidin-1-yl) methanone (1.6 g) was weighed and dissolved in 1,2-dichloroethane (20 mL), and a 2 M solution of boron tribromide in tetrahydrofuran (2.64 mL) was slowly added thereto, which were reacted at 60° C. overnight. TLC monitored that the raw materials were consumed completely, and it was quenched by addition of water, extracted with dichloromethane 2 times, dried over anhydrous sodium sulfate, and concentrated under reduced pressure to obtain the title compound (1.03 g).

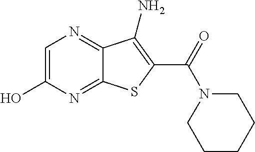 [SMILES: Nc1c(C(=O)N2CCCCC2)sc2nc(O)cnc12]

MS (ESI) m/z (M+H)⁺=279.1.

### Step 6: preparation of 7-amino-6-(piperidine-1-carbonyl) thieno[2,3-b]pyrazin-3-yltriflate

(7-Amino-3-hydroxylthieno[2,3-b]pyrazin-6-yl) (piperidin-1-yl) methanone (1.03 g) was weighed and dissolved in dichloromethane (15 mL), and N,N-diisopropylethylamine (1.23 mL), N-phenylbis(trifluoromethanesulfonyl)imide (2.64 g) were added thereto at 0° C., which were reacted at 50° C. for 3 h. TLC showed that the raw materials were consumed completely, and it was quenched by adding water, extracted twice with dichloromethane, dried over anhydrous sodium sulfate, and purified by silica gel column chromatography to obtain the title compound (878 mg).

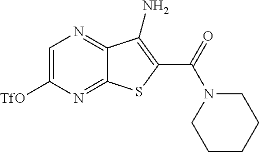 [SMILES: Nc1c(C(=O)N2CCCCC2)sc2nc(OS(=O)(=O)C(F)(F)F)cnc12]

MS (ESI) m/z (M+H)⁺=411.0.

### Step 7: preparation of (7-amino-3-(benzo[d]oxazol-6-yl) thieno[2,3-b]pyrazin-6-yl) (piperidin-1-yl) methanone

7-Amino-6-(piperidine-1-carbonyl) thieno[2,3-b]pyrazin-3-yltriflate (23 mg), benzoxazole-6-boronic acid pinacol ester (15 mg), cesium carbonate (24 mg), and [1,1′-bis(diphenylphosphino) ferrocene]dichloropalladium (3 mg) were weighed and dissolved in 1,4-dioxane (1.5 mL) and water (0.3 mL), with argon replacement three times, and reacted at 80° C. for 1.5 h. LCMS showed that the raw materials were consumed completely, and it was quenched by adding water, extracted twice with ethyl acetate, dried over anhydrous sodium sulfate, and separated by reverse phase preparative column to obtain the title compound (6.7 mg).

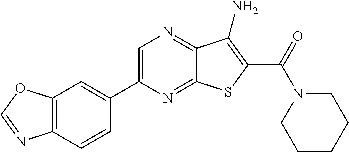 [SMILES: Nc1c(C(=O)N2CCCCC2)sc2nc(-c3ccc4ncoc4c3)cnc12]

MS (ESI) m/z (M+H)⁺=380.1,

1H NMR (400 MHZ, DMSO-d₆) δ 9.46 (s, 1H), 8.90 (d, J=1.1 Hz, 1H), 8.68 (s, 1H), 8.34 (dd, J=8.4, 1.6 Hz, 1H), 7.98 (d, J=8.5 Hz, 1H), 6.23 (s, 2H), 3.59 (t, J=5.2 Hz, 4H), 1.64 (q, J=5.5, 4.9 Hz, 2H), 1.61-1.55 (m, 4H).

### Example 3 (7-amino-3-phenylthieno[2,3-b]pyrazin-6-yl) (4-fluoropiperidin-1-yl) methanone

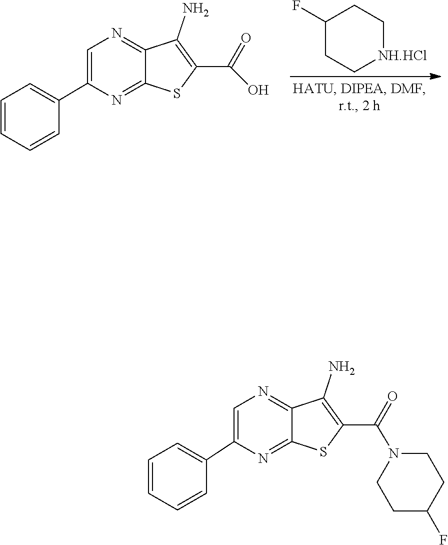

Referring to steps 1 to 3 (1) in Example 1, 7-amino-3-phenylthieno[2,3-b]pyrazine-6-carboxylic acid was prepared. 7-amino-3-phenylthieno[2,3-b]pyrazine-6-carboxylic acid (70 mg) was weighed and dissolved in N,N-dimethylformamide (2 mL), and diisopropylethylamine (130 μL), 4-fluoropiperidine hydrochloride (47 mg), 2-(7-azabenzotriazol-1-yl)-N,N,N′,N′-tetramethyluronium hexafluorophosphate (140 mg) were sequentially added thereto in an ice-water bath, which were placed and stirred at room temperature for 2 h, and the reaction was monitored by LCMS until complete. It was quenched by addition of water, and extracted with ethyl acetate three times, and the organic phases were combined, dried over anhydrous sodium sulfate, and concentrated under reduced pressure, and the residue was purified by reverse phase preparative column to obtain the title compound (12.43 mg).

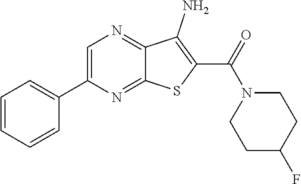 [SMILES: Nc1c(C(=O)N2CCC(F)CC2)sc2nc(-c3ccccc3)cnc12]

MS (ESI) m/z (M+H)⁺=357.1,

1H NMR (400 MHZ, DMSO-d₆) ¿ 9.34 (s, 1H), 8.25-8.23 (dd, J=8.0, 1.5 Hz, 2H), 7.60-7.54 (m, 3H), 6.27 (s, 2H), 5.00-4.85 (m, 1H), 3.72-3.57 (m, 4H), 2.02-1.90 (m, 2H), 1.82-1.77 (m, 2H).

### Examples 4-57

A series of compounds were prepared from the corresponding commercial reagents and the products in the foregoing preparation examples and Examples as raw materials, by using the preparation methods similar to that of the foregoing Examples, and the structures and characterization data of said compounds are shown in Table 1:

TABLE 1

| | | Reference | |
|---|---|---|---|
| | | Examples of | |
| | | Preparation | |
| Example | Structure | Methods | MS (M + H)⁺ & ¹H NMR |
| 4 | 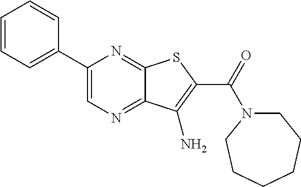 [SMILES: Nc1c(C(=O)N2CCCCCC2)sc2nc(-c3ccccc3)cnc12] | Example 1 | MS (ESI) m/z (M + H)⁺ = 353.1 ¹H NMR (400 MHZ, DMSO-d₆) δ 9.33 (s, 1H), 8.23-8.21 (d, J = 7.3 Hz, 2H), 7.58-7.51 (q, J = 8.2, 7.3 Hz, 3H), 6.40 (s, 2H), 3.66-3.63 (t, J = 5.8 Hz, 4H), 1.72 (m, 4H), 1.53 (m, 4H) |
| 5 | 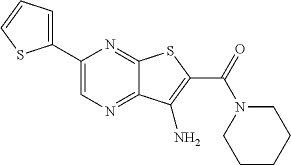 [SMILES: Nc1c(C(=O)N2CCCCC2)sc2nc(-c3cccs3)cnc12] | Example 2 | MS (ESI) m/z (M + H)⁺ = 345.0, ¹H NMR (400 MHZ, DMSO-d₆) δ 9.31 (s, 1H), 8.13 (dd, J = 3.8, 1.1 Hz, 1H), 7.83 (dd, J = 5.0, 1.1 Hz, 1H), 7.28 (dd, J = 5.0, 3.7 Hz, 1H), 6.18 (s, 2H), 3.58 (t, J = 5.4 Hz, 4H), 1.63 (t, J = 5.0 Hz, 2H), 1.60-1.54 (m, 4H) |
| 6 | 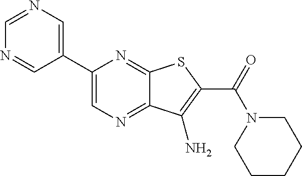 [SMILES: Nc1c(C(=O)N2CCCCC2)sc2nc(-c3cncnc3)cnc12] | Example 2 | MS (ESI) m/z (M + H)⁺ = 341.1, ¹H NMR (400 MHZ, DMSO-d₆) δ 9.59 (s, 2H), 9.48 (s, 1H), 9.34 (s, 1H), 6.24 (s, 2H), 3.59 (t, J = 5.3 Hz, 4H), 1.63 (t, J = 4.4 Hz, 2H), 1.58 (d, J = 5.0 Hz, 4H) |
| 7 | 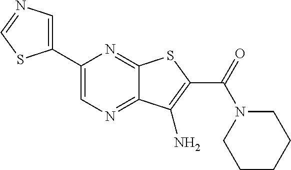 [SMILES: Nc1c(C(=O)N2CCCCC2)sc2nc(-c3cncs3)cnc12] | Example 2 | MS (ESI) m/z (M + H)⁺ = 346.1, ¹H NMR (400 MHZ, DMSO-d₆) δ 9.40 (s, 1H), 9.29 (s, 1H), 8.90 (s, 1H), 6.21 (s, 2H), 3.57 (t, J = 5.4 Hz, 4H), 1.62 (d, J = 5.1 Hz, 2H), 1.59-1.54 (m, 4H) |
| 8 | 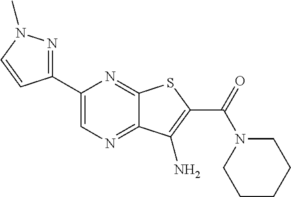 [SMILES: Cn1ccc(-c2cnc3c(N)c(C(=O)N4CCCCC4)sc3n2)n1] | Example 2 | MS (ESI) m/z (M + H)⁺ = 343.1, ¹H NMR (400 MHZ, DMSO-d₆) δ 9.20 (s, 1H), 7.89 (d, J = 2.3 Hz, 1H), 6.97 (d, J = 2.3 Hz, 1H), 6.18 (s, 2H), 3.99 (s, 3H), 3.59-3.56 (m, 4H), 1.64-1.57(m, 6H) |
| 9 | 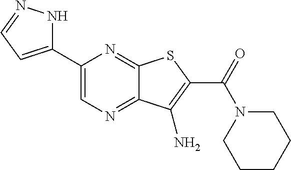 [SMILES: Nc1c(C(=O)N2CCCCC2)sc2nc(-c3ccn[nH]3)cnc12] | Example 2 | MS (ESI) m/z (M + H)⁺ = 328.9, ¹H NMR (400 MHZ, DMSO-d₆) δ 13.40 (s, 1H), 9.27 (s, 1H), 7.91 (s, 1H), 7.02 (s, 1H), 6.18 (s, 2H), 3.58 (t, J = 5.3 Hz, 4H), 1.64 (q, J = 5.3, 4.4 Hz, 2H), 1.57 (tt, J = 7.9, 4.0 Hz, 4H) |
| 10 | 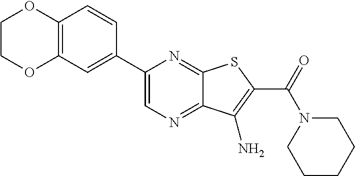 [SMILES: Nc1c(C(=O)N2CCCCC2)sc2nc(-c3ccc4c(c3)OCCO4)cnc12] | Example 2 | MS (ESI) m/z (M + H)⁺ = 397.1, ¹H NMR (400 MHZ, DMSO-d₆) δ 9.26 (s, 1H), 7.76-7.74 (m, 2H), 7.04-7.02(m, 1H), 6.18 (s, 2H), 4.33-4.32 (d, J = 1.5 Hz, 4H), 3.59-3.56 (t, J = 5.4 Hz, 4H), 1.63-1.55(m, 6H) |
| 11 | 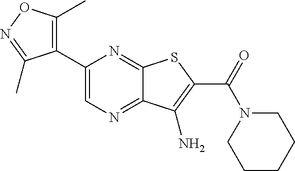 [SMILES: Cc1noc(C)c1-c1cnc2c(N)c(C(=O)N3CCCCC3)sc2n1] | Example 2 | MS (ESI) m/z (M + H)⁺ = 358.1, ¹H NMR (400 MHZ, DMSO-d₆) δ 8.88 (s, 1H), 6.19 (s, 2H), 3.58 (t, J = 5.4 Hz, 4H), 2.65 (s, 3H), 2.44 (s, 3H), 1.64 (q, J = 5.9, 4.8 Hz, 2H), 1.57 (td, J = 6.4, 3.2 Hz, 4H) |
| 12 | 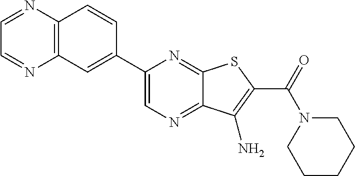 [SMILES: Nc1c(C(=O)N2CCCCC2)sc2nc(-c3ccc4nccnc4c3)cnc12] | Example 2 | MS (ESI) m/z (M + H)⁺ = 391.1, ¹H NMR (400 MHZ, DMSO-d₆) δ 9.63 (s, 1H), 9.09-8.97 (m, 3H), 8.4-8.72 (dd, J = 8.8, 2.1 Hz, 1H), 8.28-8.26(d, J = 8.8 Hz, 1H), 6.25 (s, 2H), 3.61-3.58 (t, J = 5.3 Hz, 4H), 1.64-1.58 (m, 6H) |
| 13 | 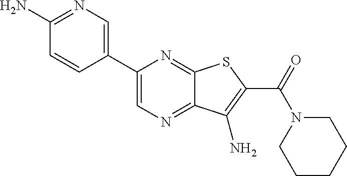 [SMILES: Nc1ccc(-c2cnc3c(N)c(C(=O)N4CCCCC4)sc3n2)cn1] | Example 2 | MS (ESI) m/z (M + H)⁺ = 355.1, ¹H NMR (400 MHZ, DMSO-d₆) δ 9.21 (s, 1H), 8.84 (d, J = 2.4 Hz, 1H), 8.23-8.20 (dd, J = 8.8, 2.5 Hz, 1H), 6.60-6.57 (m, 3H), 6.17 (s, 2H), 3.59-3.57 (t, J = 5.2 Hz, 4H), 1.66-1.54 (m, 6H) |
| 14 | 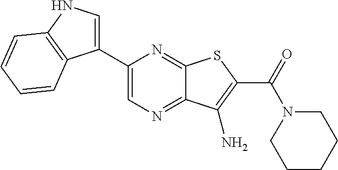 [SMILES: Nc1c(C(=O)N2CCCCC2)sc2nc(-c3c[nH]c4ccccc34)cnc12] | Example 2 | MS (ESI) m/z (M + H)⁺ = 378.1, ¹H NMR (400 MHZ, DMSO-d₆) δ 11.91 (s, 1H), 9.26 (s, 1H), 8.51 (dt, J = 4.3, 2.0 Hz, 2H), 7.53-7.49 (m, 1H), 7.23 (tt, J = 7.1, 5.5 Hz, 2H), 6.20 (s, 2H), 3.60 (t, J = 5.3 Hz, 4H), 1.64 (t, J = 4.6 Hz, 2H), 1.59 (d, J = 4.5 Hz, 4H) |
| 15 | 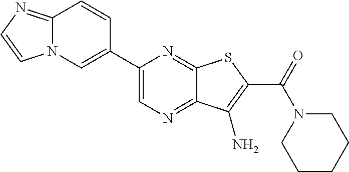 [SMILES: Nc1c(C(=O)N2CCCCC2)sc2nc(-c3ccc4nccn4c3)cnc12] | Example 2 | MS (ESI) m/z (M + H)⁺ = 379.1, ¹H NMR (400 MHZ, DMSO-d₆) δ 9.57 (s, 1H), 9.36 (s, 1H), 8.10-8.07 (d, J = 9.9 Hz, 2H), 7.75-7.73 (d, J = 9.5 Hz, 1H), 7.68 (s, 1H), 6.22 (s, 2H), 3.60-3.58 (t, J = 5.4 Hz, 4H), 1.64-1.57 (m, 6H) |
| 16 | 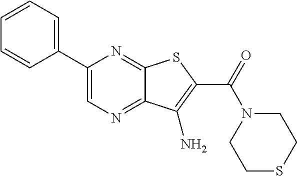 [SMILES: Nc1c(C(=O)N2CCSCC2)sc2nc(-c3ccccc3)cnc12] | Example 1 | MS (ESI) m/z (M + H)⁺ = 357.1, ¹H NMR (400 MHZ, DMSO-d₆) δ 9.35 (s, 1H), 8.26-8.24(m, 2H), 7.61-7.53 (m, 3H), 6.29 (s, 2H), 3.87-3.84 (m, 4H), 2.72-2.70 (dd, J = 6.4, 3.6 Hz, 4H) |
| 17 | 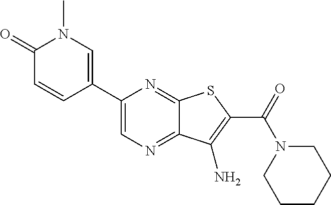 [SMILES: Cn1cc(-c2cnc3c(N)c(C(=O)N4CCCCC4)sc3n2)ccc1=O] | Example 2 | MS (ESI) m/z (M + H)⁺ = 370.1, ¹H NMR (400 MHZ, DMSO-d₆) δ 9.19 (s, 1H), 8.80 (d, J = 2.6 Hz, 1H), 8.29 (dd, J = 9.6, 2.6 Hz, 1H), 6.57 (d, J = 9.6 Hz, 1H), 6.19 (s, 2H), 3.59 (s, 3H), 3.57 (d, J = 4.6 Hz, 4H), 1.63 (d, J = 5.5 Hz, 2H), 1.60-1.54 (m, 4H) |
| 18 | 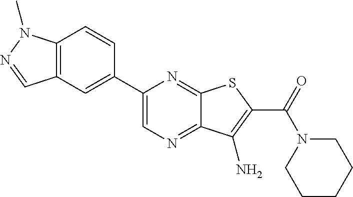 [SMILES: Cn1ncc2cc(-c3cnc4c(N)c(C(=O)N5CCCCC5)sc4n3)ccc21] | Example 2 | MS (ESI) m/z (M + H)⁺ = 393.1, ¹H NMR (400 MHZ, DMSO-d₆) δ 9.41 (s, 1H), 8.70 (d, J = 2.0 Hz, 1H), 8.30 (dd, J = 8.9, 1.7 Hz, 1H), 8.21 (d, J = 0.9 Hz, 1H), 7.82 (d, J = 8.9 Hz, 1H), 6.21 (s, 2H), 4.11 (s, 3H), 3.59 (t, J = 5.3 Hz, 4H), 1.63 (s, 2H), 1.60-1.54 (m, 4H) |
| 19 | 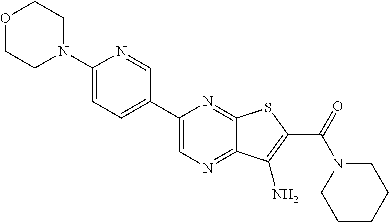 [SMILES: Nc1c(C(=O)N2CCCCC2)sc2nc(-c3ccc(N4CCOCC4)nc3)cnc12] | Example 2 | MS (ESI) m/z (M + H)⁺ = 425.1, ¹H NMR (400 MHZ, DMSO-d₆) δ 9.28 (d, J = 1.0 Hz, 1H), 9.03 (d, J = 2.4 Hz, 1H), 8.39-8.36 (dd, J = 9.1, 2.5 Hz, 1H), 7.02-7.00 (d, J = 9.0 Hz, 1H), 6.18 (s, 2H), 3.74-3.72 (t, J = 4.8 Hz, 4H), 3.62-3.57 (m, 8H), 1.64-1.56(m, 6H) |
| 20 | 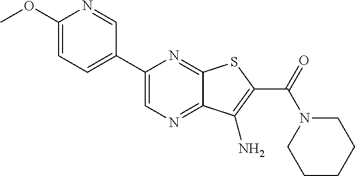 [SMILES: COc1ccc(-c2cnc3c(N)c(C(=O)N4CCCCC4)sc3n2)cn1] | Example 2 | MS (ESI) m/z (M+H)⁺ = 370.1, ¹H NMR (400 MHZ, DMSO-d₆) δ 9.43 (s, 1H), 8.36-8.35 (d, J = 5.4 Hz, 1H), 7.81-7.79 (dd, J = 5.4, 1.6 Hz, 1H), 7.64-7.63 (d, J = 1.4 Hz, 1H), 6.22 (s, 2H), 3.94 (s, 3H), 3.59-3.56 (t, J = 5.4 Hz, 4H), 1.63-1.56(m, 6H) |
| 21 | 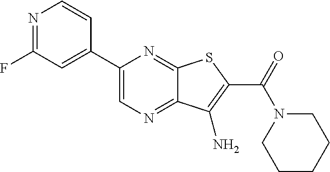 [SMILES: Nc1c(C(=O)N2CCCCC2)sc2nc(-c3ccnc(F)c3)cnc12] | Example 2 | MS (ESI) m/z (M+H)⁺ = 358.1, ¹H NMR (400 MHZ, DMSO-d₆) δ 9.52-9.51(m, 1H), 8.46-8.44 (t, J = 4.4 Hz, 1H), 8.20-8.19 (m, 1H), 8.02 (s, 1H), 6.25 (s, 2H), 3.59-3.58 (m, 4H), 1.63-1.57 (m, 6H) |
| 22 | 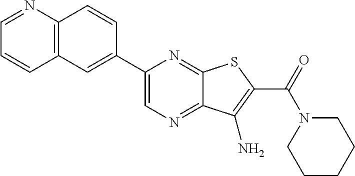 [SMILES: Nc1c(C(=O)N2CCCCC2)sc2nc(-c3ccc4ncccc4c3)cnc12] | Example 2 | MS (ESI) m/z (M+H)⁺ = 390.1, ¹H NMR (400 MHZ, DMSO-d₆) δ 9.54 (s, 1H), 9.01-8.94 (m, 2H), 8.63 (d, J = 9.0 Hz, 1H), 8.56 (d, J = 8.5 Hz, 1H), 8.19 (dt, J = 8.9, 2.1 Hz, 1H), 7.64 (dt, J = 8.2, 3.8 Hz, 1H), 6.25 (s, 2H), 3.60 (d, J = 5.6 Hz, 4H), 1.64 (s, 2H), 1.59 (s, 4H) |
| 23 | 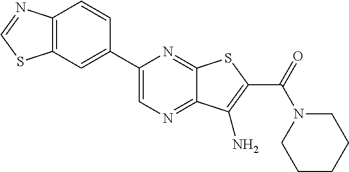 [SMILES: Nc1c(C(=O)N2CCCCC2)sc2nc(-c3ccc4ncsc4c3)cnc12] | Example 2 | MS (ESI) m/z (M + H)⁺ = 396.1, ¹H NMR (400 MHZ, DMSO-d₆) δ 9.53 (s, 1H), 9.46 (t, J= 2.2 Hz, 1H), 9.11 (d, J = 2.7 Hz, 1H), 8.44 (dd, J = 8.9, 2.6 Hz, 1H), 8.26 (dt, J = 8.6, 2.2 Hz, 1H), 6.24 (s, 2H), 3.59 (dd, J = 6.4, 3.3 Hz, 4H), 1.64 (s, 2H), 1.58 (s, 4H) |
| 24 | 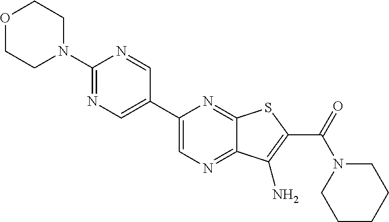 [SMILES: Nc1c(C(=O)N2CCCCC2)sc2nc(-c3cnc(N4CCOCC4)nc3)cnc12] | Example 2 | MS (ESI) m/z (M + H)⁺ = 426.1, ¹H NMR (400 MHZ, DMSO-d₆) δ 9.29 (s, 1H), 9.20 (s, 2H), 6.20 (s, 2H), 3.85-3.83 (m, 4H), 3.71-3.69 (m, 4H), 3.59-3.57 (m, 4H), 1.63-1.57 (m, 6H) |
| 25 | 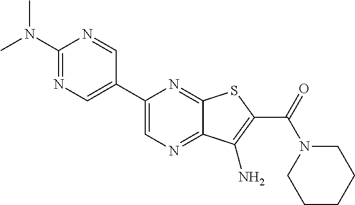 [SMILES: CN(C)c1ncc(-c2cnc3c(N)c(C(=O)N4CCCCC4)sc3n2)cn1] | Example 2 | MS (ESI) m/z (M + H)⁺ = 384.2, ¹H NMR (400 MHZ, DMSO-d₆) δ 9.27-9.26 (t, J = 2.3 Hz, 1H), 9.18-9.16 (t, J = 2.4 Hz, 2H), 6.20 (s, 2H), 3.59-3.57 (m, 4H), 3.24-3.23 (d, J = 3.0 Hz, 6H), 1.63-1.57 (m, 6H) |
| 26 | 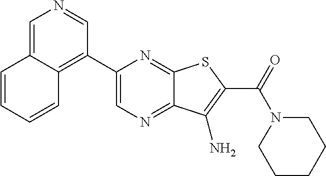 [SMILES: Nc1c(C(=O)N2CCCCC2)sc2nc(-c3cncc4ccccc34)cnc12] | Example 2 | MS (ESI) m/z (M + H)⁺ = 390.1, ¹H NMR (400 MHZ, DMSO-d₆) δ 9.49 (t, J = 1.9 Hz, 1H), 9.12 (t, J = 2.0 Hz, 1H), 8.82 (t, J = 2.0 Hz, 1H), 8.28 (td, J = 8.1, 2.3 Hz, 2H), 7.91-7.85 (m, 1H), 7.80 (td, J = 7.9, 2.3 Hz, 1H), 6.28 (s, 2H), 3.61 (t, J = 4.5 Hz, 4H), 1.65 (d, J = 6.9 Hz, 2H), 1.62-1.52 (m, 4H) |
| 27 | 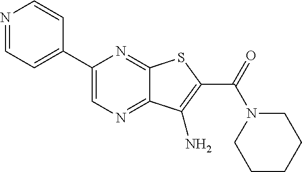 [SMILES: Nc1c(C(=O)N2CCCCC2)sc2nc(-c3ccncc3)cnc12] | Example 2 | MS (ESI) m/z (M + H)⁺ = 340.1, ¹H NMR (400 MHZ, DMSO-d₆) δ 9.46 (s, 1H), 8.79-8.77 (m, 2H), 8.22-8.20 (m, 2H), 6.23 (s, 2H), 3.60-3.57 (t, J = 5.2 Hz, 4H), 1.64-1.56 (m, 6H) |
| 28 | 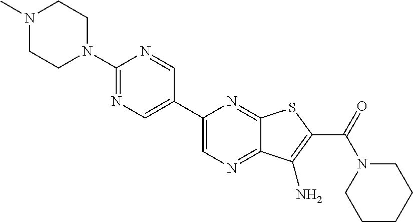 [SMILES: CN1CCN(c2ncc(-c3cnc4c(N)c(C(=O)N5CCCCC5)sc4n3)cn2)CC1] | Example 2 | MS (ESI) m/z (M + H)⁺ = 439.2, ¹H NMR (400 MHZ, DMSO-d₆) δ 9.28 (d, J = 1.5 Hz, 1H), 9.18 (d, J = 1.5 Hz, 2H), 6.20 (s, 2H), 3.88-3.84 (t, J = 5.2 Hz, 4H), 3.59-3.56 (t, J = 5.3 Hz, 4H), 2.43-2.40 (m, 4H), 2.25 (s, 3H), 1.64-1.55 (m, 6H) |
| 29 | 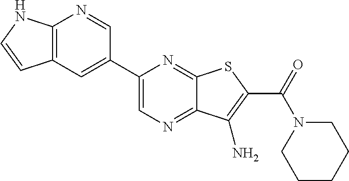 [SMILES: Nc1c(C(=O)N2CCCCC2)sc2nc(-c3cnc4[nH]ccc4c3)cnc12] | Example 2 | MS (ESI) m/z (M + H)⁺ = 379.1, ¹H NMR (400 MHZ, DMSO-d₆) δ 11.94 (s, 1H), 9.41 (s, 1H), 9.10 (s, 1H), 8.81 (s, 1H), 7.59 (s, 1H), 6.61 (s, 1H), 6.22 (s, 2H), 3.59 (s, 4H), 1.64 (s, 2H), 1.62-1.53 (m, 4H) |
| 30 | 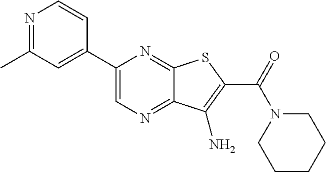 [SMILES: Cc1cc(-c2cnc3c(N)c(C(=O)N4CCCCC4)sc3n2)ccn1] | Example 2 | MS (ESI) m/z (M + H)⁺ = 354.1, ¹H NMR (400 MHZ, DMSO-d₆) δ 9.43 (s, 1H), 8.63 (d, J = 5.3 Hz, 1H), 8.09 (d, J = 1.6 Hz, 1H), 8.00 (dd, J = 5.3, 1.7 Hz, 1H), 6.23 (s, 2H), 3.58 (t, J = 5.3 Hz, 4H), 2.60 (s, 3H), 1.63 (t, J = 4.8 Hz, 2H), 1.57 (dq, J = 6.6, 3.2 Hz, 4H) |
| 31 | 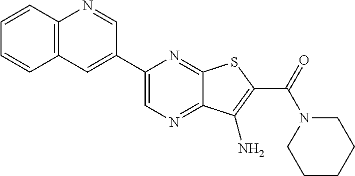 [SMILES: Nc1c(C(=O)N2CCCCC2)sc2nc(-c3cnc4ccccc4c3)cnc12] | Example 2 | MS (ESI) m/z (M + H)⁺ = 390.1, ¹H NMR (400 MHZ, DMSO-d₆) δ 9.74 (d, J = 2.3 Hz, 1H), 9.58 (s, 1H), 9.26 (d, J = 2.3 Hz, 1H), 8.23-8.16 (m, 1H), 8.12 (dd, J = 8.4, 1.1 Hz, 1H), 7.87 (d, J = 1.6 Hz, 1H), 7.73 (d, J = 8.1 Hz, 1H), 6.26 (s, 2H), 3.60 (t, J = 5.3 Hz, 4H), 1.64 (t, J = 4.5 Hz, 2H), 1.62-1.53 (m, 4H) |
| 32 | 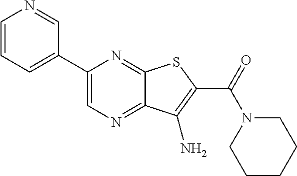 [SMILES: Nc1c(C(=O)N2CCCCC2)sc2nc(-c3cccnc3)cnc12] | Example 2 | MS (ESI) m/z (M + H)⁺ = 340.1, ¹H NMR (400 MHZ, DMSO-d₆) δ 9.42 (d, J = 2.8 Hz, 2H), 8.73 (dd, J = 4.7, 1.6 Hz, 1H), 8.60 (dt, J = 8.0, 2.0 Hz, 1H), 7.61 (dd, J = 8.0, 4.8 Hz, 1H), 6.23 (s, 2H), 3.59 (t, J = 5.3 Hz, 4H), 1.67-1.62 (m, 2H), 1.58 (d, J = 4.8 Hz, 4H) |
| 33 | 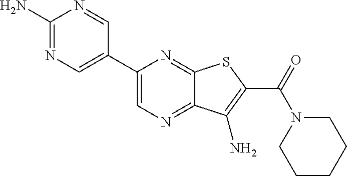 [SMILES: Nc1ncc(-c2cnc3c(N)c(C(=O)N4CCCCC4)sc3n2)cn1] | Example 2 | MS (ESI) m/z (M + H)⁺ = 356.1, ¹H NMR (400 MHZ, DMSO-d₆) δ 9.25 (s, 1H), 9.08 (s, 2H), 7.26 (s, 2H), 6.19 (s, 2H), 3.59-3.56 (m, 4H), 1.64-1.56 (m, 6H) |
| 34 | 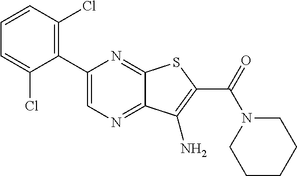 [SMILES: Nc1c(C(=O)N2CCCCC2)sc2nc(-c3c(Cl)cccc3Cl)cnc12] | Example 2 | MS (ESI) m/z (M + H)⁺ = 407.1, ¹H NMR (400 MHZ, DMSO-d₆) δ 8.87 (s, 1H), 7.71 (d, J = 1.2 Hz, 1H), 7.69 (d, J = 0.6 Hz, 1H), 7.61 (dd, J = 9.0, 7.2 Hz, 1H), 6.24 (s, 2H), 3.59 (t, J = 5.3 Hz, 4H), 1.67-1.61 (m, 2H), 1.58 (d, J = 6.1 Hz, 4H) |
| 35 | 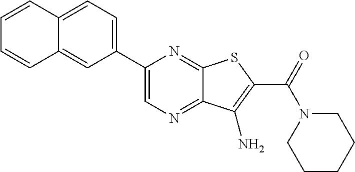 [SMILES: Nc1c(C(=O)N2CCCCC2)sc2nc(-c3ccc4ccccc4c3)cnc12] | Example 2 | MS (ESI) m/z (M + H)⁺ = 3 89.1, ¹H NMR (400 MHZ, DMSO-d₆) δ 9.52 (s, 1H), 8.89 (d, J = 1.9 Hz, 1H), 8.39 (dd, J = 8.6, 1.8 Hz, 1H), 8.15-8.10 (m, 2H), 8.01 (dd, J = 6.1, 3.4 Hz, 1H), 7.62 (dt, J = 6.3, 3.5 Hz, 2H), 6.24 (s, 2H), 3.60 (t, J = 5.3 Hz, 4H), 1.65 (q, J = 5.9, 4.4 Hz, 2H), 1.62-1.55 (m, 4H) |
| 36 | 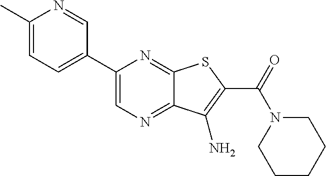 [SMILES: Cc1ccc(-c2cnc3c(N)c(C(=O)N4CCCCC4)sc3n2)cn1] | Example 2 | MS (ESI) m/z (M + H)⁺ = 354.1, ¹H NMR (400 MHZ, DMSO-d₆) δ 9.39 (s, 1H), 9.29 (d, J = 2.4 Hz, 1H), 8.49 (dd, J = 8.1, 2.4 Hz, 1H), 7.46 (d, J = 8.1 Hz, 1H), 6.22 (s, 2H), 3.58 (t, J = 5.3 Hz, 4H), 2.57 (s, 3H), 1.67-1.61 (m, 2H), 1.58 (d, J = 5.3 Hz, 4H) |
| 37 | 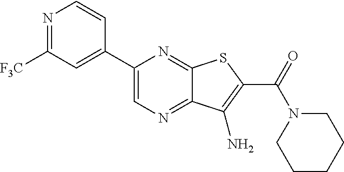 [SMILES: Nc1c(C(=O)N2CCCCC2)sc2nc(-c3ccnc(C(F)(F)F)c3)cnc12] | Example 2 | MS (ESI) m/z (M + H)⁺ = 408.1, ¹H NMR (400 MHZ, DMSO-d₆) δ 9.61 (s, 1H), 8.98 (d, J = 5.1 Hz, 1H), 8.69-8.65 (m, 1H), 8.56 (dd, J = 5.1, 1.7 Hz, 1H), 6.26 (s, 2H), 3.59 (t, J = 5.3 Hz, 4H), 1.64 (d, J = 4.5 Hz, 2H), 1.58 (d, J = 6.4 Hz, 4H) |
| 38 | 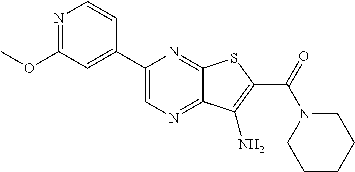 [SMILES: COc1cc(-c2cnc3c(N)c(C(=O)N4CCCCC4)sc3n2)ccn1] | Example 2 | MS (ESI) m/z (M + H)⁺ = 370.1, ¹H NMR (400 MHZ, DMSO-d₆) δ 9.43 (s, 1H), 8.36-8.35 (d, J = 5.4 Hz, 1H), 7.80-7.79 (dd, J = 5.3, 1.5 Hz, 1H), 7.63 (s, 1H), 6.22 (s, 2H), 3.94 (s, 3H), 3.58-3.57 (t, J = 5.3 Hz, 4H), 1.66-1.54 (m, 6H) |
| 39 | 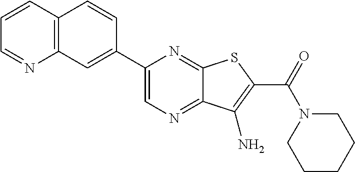 [SMILES: Nc1c(C(=O)N2CCCCC2)sc2nc(-c3ccc4cccnc4c3)cnc12] | Example 2 | MS (ESI) m/z (M + H)⁺ = 390.1, ¹H NMR (400 MHZ, DMSO-d₆) δ 9.60 (s, 1H), 9.01 (dd, J = 4.2, 1.8 Hz, 1H), 8.92 (d, J = 1.7 Hz, 1H), 8.48 (ddd, J = 8.5, 6.7, 1.8 Hz, 2H), 8.19 (d, J = 8.6 Hz, 1H), 7.63 (dd, J = 8.3, 4.2 Hz, 1H), 6.25 (s, 2H), 3.60 (t, J = 5.2 Hz, 4H), 1.65 (q, J = 5.3, 4.7 Hz, 2H), 1.62-1.54 (m, 4H) |
| 40 | 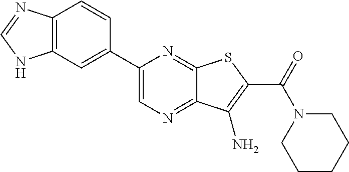 [SMILES: Nc1c(C(=O)N2CCCCC2)sc2nc(-c3ccc4nc[nH]c4c3)cnc12] | Example 2 | MS (ESI) m/z (M + H)⁺ = 379.1, ¹H NMR (400 MHZ, DMSO-d₆) δ 12.71 (d, J = 15.2 Hz, 1H), 9.40 (d, J = 15.0 Hz, 1H), 8.58-8.40 (m, 1H), 8.36 (d, J = 8.3 Hz, 1H), 8.15-8.10 (m, 1H), 7.81-7.71 (m, 1H), 6.21 (s, 2H), 3.60 (t, J = 5.3 Hz, 4H), 1.64 (s, 2H), 1.61-1.55 (m, 4H). |
| 41 | 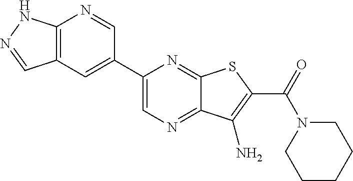 [SMILES: Nc1c(C(=O)N2CCCCC2)sc2nc(-c3cnc4[nH]ncc4c3)cnc12] | Example 2 | MS (ESI) m/z (M + H)⁺ = 380.1, ¹H NMR (400 MHz, DMSO-d₆) δ 13.92 (s, 1H), 9.46 (s, 1H), 9.38 (d, J = 2.2 Hz, 1H), 9.09 (s, 1H), 8.31 (s, 1H), 6.24 (s, 2H), 3.60 (d, J = 5.2 Hz, 4H), 1.62 (d, J = 24.1 Hz, 6H) |
| 42 | 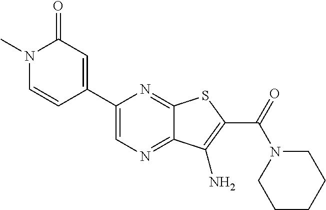 [SMILES: Cn1ccc(-c2cnc3c(N)c(C(=O)N4CCCCC4)sc3n2)cc1=O] | Example 2 | MS (ESI) m/z (M + H)⁺ = 370.1, ¹H NMR (400 MHZ, DMSO-d₆) δ 9.38 (s, 1H), 7.89 (d, J = 7.1 Hz, 1H), 7.31 (d, J = 2.0 Hz, 1H), 7.03 (dd, J = 7.1, 2.1 Hz, 1H), 6.22 (s, 2H), 3.58 (t, J = 5.3 Hz, 4H), 3.50 (s, 3H), 1.63 (d, J = 5.3 Hz, 2H), 1.60-1.54 (m, 4H) |
| 43 | 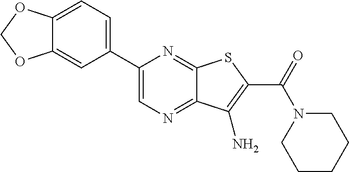 [SMILES: Nc1c(C(=O)N2CCCCC2)sc2nc(-c3ccc4c(c3)OCO4)cnc12] | Example 2 | MS (ESI) m/z (M + H)⁺ = 383.1, ¹H NMR (400 MHZ, DMSO-d₆) δ 9.27 (s, 1H), 7.84-7.82 (dd, J = 8.1, 1.8 Hz, 1H), 7.80-7.79 (d, J = 1.7 Hz, 1H), 7.12-7.10 (d, J = 8.1 Hz, 1H), 6.19 (s, 2H), 6.14 (s, 2H), 3.59-3.57 (t, J = 5.3 Hz, 4H), 1.66-1.55 (m, 6H) |
| 44 | 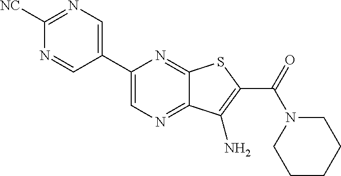 [SMILES: N#Cc1ncc(-c2cnc3c(N)c(C(=O)N4CCCCC4)sc3n2)cn1] | Example 2 | MS (ESI) m/z (M + H)⁺ = 3 66.1, ¹H NMR (400 MHZ, DMSO-d₆) δ 9.80 (s, 2H), 9.57 (s, 1H), 6.27 (s, 2H), 3.58 (t, J = 5.3 Hz, 4H), 1.64 (d, J = 5.1 Hz, 2H), 1.60-1.55 (m, 4H) |
| 45 | 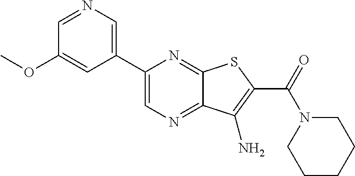 [SMILES: COc1cncc(-c2cnc3c(N)c(C(=O)N4CCCCC4)sc3n2)c1] | Example 2 | MS (ESI) m/z (M + H)⁺ = 370.1, ¹H NMR (400 MHZ, DMSO-d₆) δ 9.45 (s, 1H), 9.03 (d, J = 1.8 Hz, 1H), 8.45 (d, J = 2.8 Hz, 1H), 8.12 (dd, J= 2.9, 1.8 Hz, 1H), 6.23 (s, 2H), 3.97 (s, 3H), 3.59 (t, J = 5.3 Hz, 4H), 1.68-1.62 (m, 2H), 1.58 (d, J = 6.3 Hz, 4H) |
| 46 | 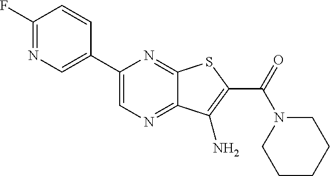 [SMILES: Nc1c(C(=O)N2CCCCC2)sc2nc(-c3ccc(F)nc3)cnc12] | Example 2 | MS (ESI) m/z (M + H)⁺ = 358.1, ¹H NMR (400 MHZ, DMSO-d₆) δ 9.41 (s, 1H), 9.10-9.09 (d, J = 2.5 Hz, 1H), 8.81-8.77 (td, J = 8.2, 2.6 Hz, 1H), 7.43-7.40 (dd, J = 8.7, 2.8 Hz, 1H), 6.23 (s, 2H), 3.60-3.57(t, J = 5.3 Hz, 4H), 1.66-1.55 (m, 6H) |
| 47 | 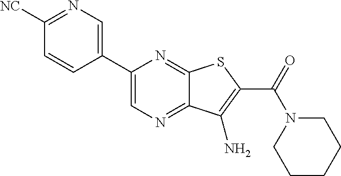 [SMILES: N#Cc1ccc(-c2cnc3c(N)c(C(=O)N4CCCCC4)sc3n2)cn1] | Example 2 | MS (ESI) m/z (M + H)⁺ = 365.1, ¹H NMR (400 MHZ, DMSO-d₆) δ 9.58 (dd, J = 2.3, 0.8 Hz, 1H), 9.52 (s, 1H), 8.86-8.83 (dd, J = 8.2, 2.3 Hz, 1H), 8.26-8.24 (dd, J = 8.2, 0.9 Hz, 1H), 6.25 (s, 2H), 3.60-3.57 (t, J = 5.4 Hz, 4H), 1.64-1.57 (m, 6H) |
| 48 | 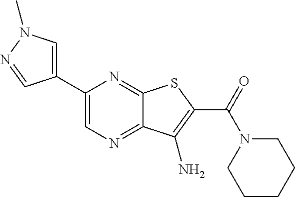 [SMILES: Cn1cc(-c2cnc3c(N)c(C(=O)N4CCCCC4)sc3n2)cn1] | Example 2 | MS (ESI) m/z (M + H)⁺ = 343.1, ¹H NMR (400 MHZ, DMSO-d₆) δ 9.05 (s, 1H), 8.56 (s, 1H), 8.21 (d, J = 0.7 Hz, 1H), 6.16 (s, 2H), 3.93 (s, 3H), 3.57 (t, J = 5.3 Hz, 4H), 1.63 (q, J = 5.1 Hz, 2H), 1.56 (d, J = 4.7 Hz, 4H) |
| 49 | 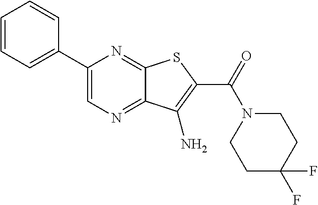 [SMILES: Nc1c(C(=O)N2CCC(F)(F)CC2)sc2nc(-c3ccccc3)cnc12] | Example 1 | MS (ESI) m/z (M + H)⁺ = 375.1, ¹H NMR (400 MHZ, DMSO-d₆) δ 9.36 (s, 1H), 8.26-8.24 (dd, J = 8.0, 1.5 Hz, 2H), 7.61-7.55 (m, 3H), 6.34 (s, 2H), 3.72-3.69 (m, 4H), 2.16-2.06 (m, 4H) |
| 50 | 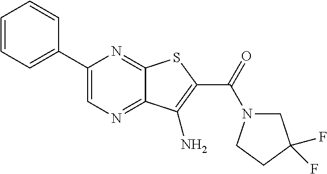 [SMILES: Nc1c(C(=O)N2CCC(F)(F)C2)sc2nc(-c3ccccc3)cnc12] | Example 1 | MS (ESI) m/z (M + H)⁺ = 361.1, ¹H NMR (400 MHZ, DMSO-d₆) δ 9.39 (s, 1H), 8.27-8.25(m, 2H), 7.62-7.56 (m, 3H), 6.98 (s, 2H), 4.11-4.05 (t, J = 13.1 Hz, 2H), 3.96-3.92 (t, J = 7.3 Hz, 2H), 2.60-2.54 (m, 2H) |
| 51 | 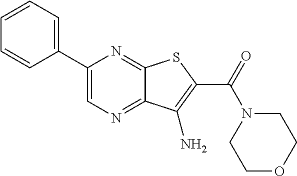 [SMILES: Nc1c(C(=O)N2CCOCC2)sc2nc(-c3ccccc3)cnc12] | Example 1 | MS (ESI) m/z (M + H)⁺ = 341.1, ¹H NMR (400 MHZ, DMSO-d₆) δ 9.35 (d, J = 1.1 Hz, 1H), 8.6-8.23 (m, 2H), 7.61-7.53 (m, 3H), 6.31 (s, 2H), 3.69-3.61 (m, 8H) |
| 52 | 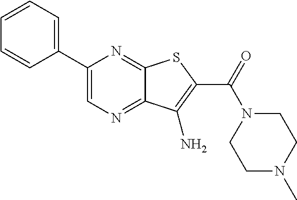 [SMILES: CN1CCN(C(=O)c2sc3nc(-c4ccccc4)cnc3c2N)CC1] | Example 1 | MS (ESI) m/z (M + H)⁺ = 354.0, ¹H NMR (400 MHZ, DMSO-d₆) δ 9.35 (d, J = 1.1 Hz, 1H), 8.26-8.23 (dt, J = 7.9, 1.5 Hz, 2H), 7.61-7.53 (m, 3H), 6.26 (s, 2H), 3.64-3.61 (t, J = 4.9 Hz, 4H), 2.39-2.36 (t, J = 5.0 Hz, 4H), 2.21 (s, 3H) |
| 53 | 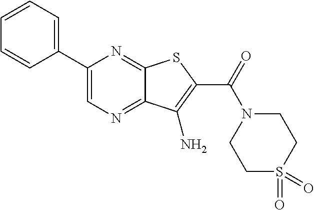 [SMILES: Nc1c(C(=O)N2CCS(=O)(=O)CC2)sc2nc(-c3ccccc3)cnc12] | Example 1 | MS (ESI) m/z (M + H)⁺ = 389.1, ¹H NMR (400 MHZ, DMSO-d₆) δ 9.36 (d, J = 1.1 Hz, 1H), 8.7-8.24 (m, 2H), 7.61-7.54 (m, 3H), 6.41 (s, 2H), 4.01-3.98 (t, J = 5.1 Hz, 4H), 3.33-3.29 (m, 4H) |
| 54 | 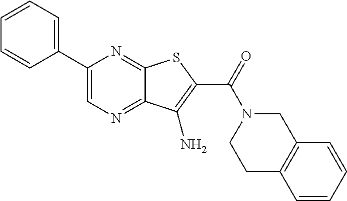 [SMILES: Nc1c(C(=O)N2CCc3ccccc3C2)sc2nc(-c3ccccc3)cnc12] | Example 1 | MS (ESI) m/z (M + H)⁺ = 387.1, ¹H NMR (400 MHZ, DMSO-d₆) δ 9.37 (s, 1H), 8.27-8.25 (m, 2H), 7.62-7.53 (m, 3H), 7.20 (s, 4H), 6.42 (s, 2H), 4.82 (s, 2H), 3.90-3.87 (t, J = 5.9 Hz, 2H), 2.96-2.93 (t, J = 6.0 Hz, 2H) |
| 55 | 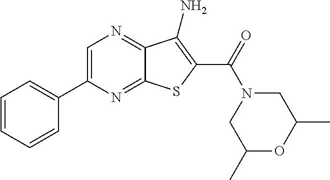 [SMILES: CC1CN(C(=O)c2sc3nc(-c4ccccc4)cnc3c2N)CC(C)O1] | Example 1 | MS (ESI) m/z (M + H)⁺ = 369.1, ¹H NMR (400 MHZ, DMSO-d₆) δ 9.35 (s, 1H), 8.25-8.23 (d, J = 7.3 Hz, 2H), 7.60-7.53 (m, 3H), 6.29 (s, 2H), 4.07-4.04 (d, J = 12.9 Hz, 2H), 3.66-3.58 (m, 2H), 2.80-2.74 (t, J = 11.8 Hz, 2H), 1.12-1.10 (d, J = 6.1 Hz, 6H) |
| 56 | 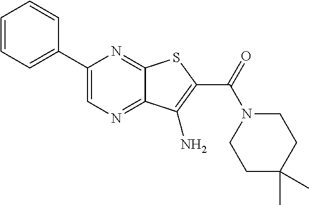 [SMILES: CC1(C)CCN(C(=O)c2sc3nc(-c4ccccc4)cnc3c2N)CC1] | Example 1 | MS (ESI) m/z (M + H)⁺ = 367.2, ¹H NMR (400 MHZ, DMSO-d₆) δ 9.34 (d, J = 1.4 Hz, 1H), 8.25-8.23 (d, J = 7.3 Hz, 2H), 7.60-7.54 (m, 3H), 6.21 (s, 2H), 3.62-3.59 (dd, J = 7.2, 4.3 Hz, 4H), 1.39-1.36 (dd, J = 7.2, 4.3 Hz, 4H), 0.99 (s, 6H) |
| 57 | 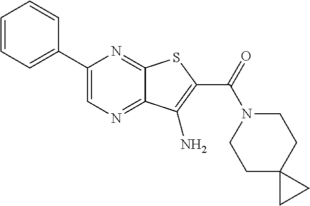 [SMILES: Nc1c(C(=O)N2CCC3(CC2)CC3)sc2nc(-c3ccccc3)cnc12] | Example 1 | MS (ESI) m/z (M + H)⁺ = 365.1, ¹H NMR (400 MHZ, DMSO-d₆) δ 9.34 (d, J = 1.4 Hz, 1H), 8.26-8.23 (dd, J = 5.6, 3.7 Hz, 2H), 7.61-7.52 (m, 3H), 6.23 (s, 2H), 3.67-3.64 (dd, J = 6.8, 4.1 Hz, 4H), 1.43-1.40 (dd, J = 6.8, 4.1 Hz, 4H), 0.37 (s, 4H) |
| 58 | 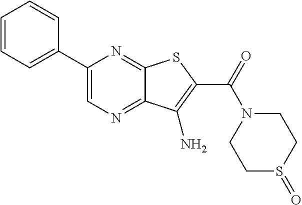 [SMILES: Nc1c(C(=O)N2CCS(=O)CC2)sc2nc(-c3ccccc3)cnc12] | Example 1 | MS (ESI) m/z (M + H)⁺ = 373.1, ¹H NMR (400 MHZ, DMSO-d₆) δ 9.36 (d, J = 1.2 Hz, 1H), 8.26-8.24 (d, J = 7.2 Hz, 2H), 7.58 (q, J = 7.2 Hz, 3H), 6.36 (s, 2H), 4.10-4.05 (m, 2H), 3.94-3.87 (t, J = 12.8 Hz, 2H), 3.06-2.99 (m, 2H), 2.87-2.84 (m, 2H) |
| 59 | 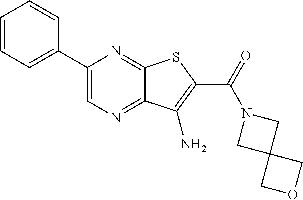 [SMILES: Nc1c(C(=O)N2CC3(COC3)C2)sc2nc(-c3ccccc3)cnc12] | Example 1 | MS (ESI) m/z (M + H)⁺ = 353.1, ¹H NMR (400 MHZ, DMSO-d₆) δ 9.39 (s, 1H), 8.27-8.25 (m, 2H), 7.60-7.56 (m, 3H), 7.09 (s, 2H), 4.73 (s, 4H), 4.45 (s, 4H) |
| 60 | 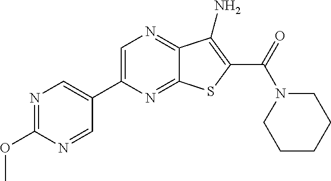 [SMILES: COc1ncc(-c2cnc3c(N)c(C(=O)N4CCCCC4)sc3n2)cn1] | Example 2 | MS (ESI) m/z (M + H)⁺ = 371.0, ¹H NMR (400 MHZ, DMSO-d₆) δ 9.40 (s, 2H), 9.39 (s, 1H), 6.23 (s, 2H), 4.03 (s, 3H), 3.58 (t, J = 5.3 Hz, 4H), 1.63 (d, J = 8.2 Hz, 2H), 1.60-1.55 (m, 4H) |
| 61 | 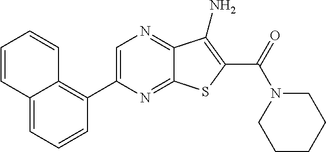 [SMILES: Nc1c(C(=O)N2CCCCC2)sc2nc(-c3cccc4ccccc34)cnc12] | Example 2 | MS (ESI) m/z (M + H)⁺ = 389.0, ¹H NMR (400 MHZ, DMSO-d₆) δ 9.01 (s, 1H), 8.13 (dt, J = 8.2, 1.3 Hz, 2H), 8.09-8.06 (m, 1H), 7.81 (dd, J = 7.1, 1.3 Hz, 1H), 7.69 (dd, J = 8.2, 7.1 Hz, 1H), 7.63 - 7.55 (m, 2H), 6.26 (s, 2H), 3.61 (t, J = 5.2 Hz, 4H), 1.64 (q, J = 5.2, 4.3 Hz, 2H), 1.59 (q, J = 5.6, 4.7 Hz, 4H) |
| 62 | 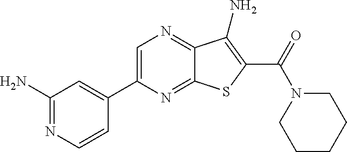 [SMILES: Nc1cc(-c2cnc3c(N)c(C(=O)N4CCCCC4)sc3n2)ccn1] | Example 2 | MS (ESI) m/z (M + H)⁺ = 355.1, ¹H NMR (400 MHZ, DMSO-d₆) δ 9.26 (s, 1H), 8.11-8.07 (m, 1H), 7.30-7.27 (m, 2H), 6.20 (d, J = 9.6 Hz, 4H), 3.58 (t, J = 5.3 Hz, 4H), 1.63 (q, J = 5.3, 4.7 Hz, 2H), 1.57 (q, J = 5.2, 4.2 Hz, 4H) |
| 63 | 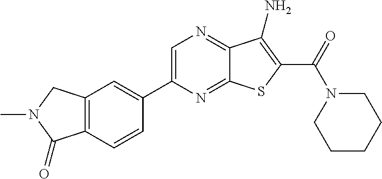 [SMILES: CN1Cc2cc(-c3cnc4c(N)c(C(=O)N5CCCCC5)sc4n3)ccc2C1=O] | Example 2 | MS (ESI) m/z (M + H)⁺ = 408.1, ¹H NMR (400 MHZ, DMSO-d₆) δ 9.41 (s, 1H), 8.46 (s, 1H), 8.35 (d, J = 8.2 Hz, 1H), 7.83 (d, J = 8.0 Hz, 1H), 6.22 (s, 2H), 4.58 (s, 2H), 3.60-3.57 (m, 4H), 3.12 (s, 3H), 1.64-1.57 (m, 6H) |
| 64 | 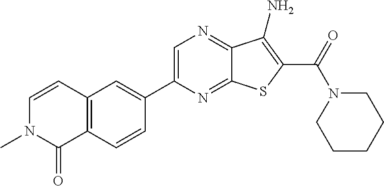 [SMILES: Cn1ccc2cc(-c3cnc4c(N)c(C(=O)N5CCCCC5)sc4n3)ccc2c1=O] | Example 2 | MS (ESI) m/z (M + H)⁺ = 420.1, ¹H NMR (400 MHZ, DMSO-d₆) δ 9.46 (s, 1H), 8.56 (s, 1H), 8.38-8.32 (m, 2H), 7.57-7.55 (d, J= 7.3 Hz, 1H), 6.79-6.77 (d, J= 7.3 Hz, 1H), 6.22 (s, 2H), 3.62-3.58 (m, 4H), 3.54 (s, 3H), 1.64-1.57 (m, 6H) |

### Biological Tests

### Test Example 1: Detection of 15-PGDH Enzyme Activity

### 1. Experimental Materials:

| Reagents/Materials/Instruments | Manufacturer | Item No. / Model No. |
|---|---|---|
| 15-PGDH | Sino Biological Inc. | 11205-H08E |
| β-NAD | Sigma-Aldrich Corporation | N6522 |
| PGF2α | MedChemExpress LLC | HY-12956A |
| DMSO | Sigma-Aldrich Corporation | D8418 |
| 384-well plate | Corning United States Corporation | 4513 |
| Tween 20 | Shanghai Macklin Biochemical | T818927 |
| | Technology Co., Ltd | |
| Tris-HCl | Shanghai Beyotime Biotech Inc. | ST774 |
| Multifunctional microplate reader | BMG LABTECH Corporation | PHERAstar ® FSX |

### 2. Experimental Method:

a. A solution of pH 7.5 containing 50 mM Tris-HCl, 0.01% Tween 20 was prepared with ultrapure water as a reaction buffer;
b. A 10 mM mother liquor of the compound to be tested was prepared with DMSO, and then the reaction buffer was used to dilute the mother liquor of the compound to be tested to obtain solution 1 of the compound to be tested at a concentration of 40,000 nM, and then the solution 1 of the compound to be tested was serially diluted into solutions 2-9 (or 2-12) of the compound to be tested at 9 (or 11) concentrations with a gradient difference of three-fold. 5 μL of each concentration of solutions of the compound to be tested was respectively taken and added into a 384-well plate as test wells;
c. Then 5 μL of the reaction buffer was added to the blank wells of the 384-well plate as positive control and blank control wells, respectively;
d. The reaction buffer was used to prepare a 15-PGDH protein solution at a concentration of 5 ng/μL, 5 μL of the 15-PGDH protein solution was taken and added to the test wells and positive control wells, and meanwhile another 5 μL of the reaction buffer was added to the blank control wells, then the plate was centrifuged at 2000 rpm for 30 seconds;
e. The reaction buffer was used to prepare 5 mM β-NAD and 2 mM PGF2α, respectively, which were mixed at 1:1 by volume to obtain a substrate mixture, 10 μL of the substrate mixture was taken and added to the test wells, positive control wells and blank control wells to start the reaction;
f. The fluorescence signal value (Ex/Em=340/450) of each well was detected continuously by using a multifunctional microplate reader.

### 3. Data Analysis:

a) Continuous fluorescence signal values were analyzed by using the “kinetic calculations-slope calculation method” in the PHERAstar Data analysis software to obtain the slope of each test well;
b) The inhibition rate % was calculated by using the following formula:
   inhibition rate %=[1-(slope of test well-signal value of positive control well)/(signal value of blank control well-average signal value of positive control well)]×100%.
c) Calculation of IC₅₀ and plotting of inhibition rate-dose curves: IC₅₀ values were calculated by fitting compound concentrations and corresponding inhibition rates with a nonlinear regression (dose response-variable slope) via using GraphPad Prism 6.0.

The formula was shown below: Y=Bottom+(Top-Bottom)/(1+10∧((LogIC50−X)*HillSlope)), wherein X is a log value of a concentration of the compound, and Y is inhibition rate %.

### 4. Experimental Results:

The compounds of the present application had the following inhibitory activities on 15-PGDH enzyme:

| Example No. | IC₅₀ (nM) |
|---|---|
| Example 1 | C |
| Example 2 | B |
| Example 3 | D |
| Example 4 | D |
| Example 5 | B |
| Example 6 | B |
| Example 7 | B |
| Example 8 | C |
| Example 9 | C |
| Example 10 | B |
| Example 11 | B |
| Example 12 | B |
| Example 13 | B |
| Example 14 | / |
| Example 15 | B |
| Example 16 | D |
| Example 17 | B |
| Example 18 | C |
| Example 19 | C |
| Example 20 | B |
| Example 21 | A |
| Example 22 | B |
| Example 23 | A |
| Example 24 | A |
| Example 25 | A |
| Example 26 | B |
| Example 27 | B |
| Example 28 | B |
| Example 29 | B |
| Example 30 | B |
| Example 31 | B |
| Example 32 | C |
| Example 33 | A |
| Example 34 | B |
| Example 35 | B |
| Example 36 | B |
| Example 37 | C |
| Example 38 | C |
| Example 39 | B |
| Example 40 | B |
| Example 41 | C |
| Example 42 | B |
| Example 43 | C |
| Example 44 | C |
| Example 45 | B |
| Example 46 | B |
| Example 47 | A |
| Example 48 | D |
| Example 49 | E |
| Example 50 | E |
| Example 51 | E |
| Example 52 | E |
| Example 53 | F |
| Example 54 | E |
| Example 55 | E |
| Example 56 | E |
| Example 57 | E |
| Example 58 | G |
| Example 59 | G |
| Example 60 | A |
| Example 61 | C |
| Example 62 | B |
| Example 63 | B |
| Example 64 | B |

In the table, “/” represents not detected; “A” represents that the IC₅₀ range of 15-PGDH enzyme inhibitory activity is less than 0.5 nM; “B” represents that the IC₅₀ range of 15-PGDH enzyme inhibitory activity is greater than or equal to 0.5 nM and less than 3 nM; “C” represents that the IC₅₀ range of 15-PGDH enzyme inhibitory activity is greater than or equal to 3 nM and less than 10 nM; “D” represents that the IC₅₀ range of 15-PGDH enzyme inhibitory activity is greater than or equal to 10 nM and less than or equal to 25 nM; “E” represents that the IC₅₀ range of 15-PGDH enzyme inhibitory activity is greater than or equal to 50 nM and less than or equal to 1000 nM; “F” represents that 15-PGDH enzyme inhibitory activity is greater than 1000 nM and less than or equal to 5000 nM; and “G” represents that 15-PGDH enzyme inhibitory activity is inactive (IC₅₀>5000 nM).

The results showed that the compounds of the present application can have strong inhibitory activity against 15-PGDH enzyme.

### Test Example 2: Assay of Intracellular PGE2 Up-Regulatory Activity

### 1. Experimental Materials:

| Reagents/Materials/Instruments | Manufacturer | Item No. |
|---|---|---|
| F12k Kaighn's Modification culture | Hyclone Laboratories, Inc | SH3052601/AG29722854 |
| medium | | |
| TRYPSIN | Hyclone Laboratories, Inc | J190002 |
| Fetal Bovine Serum | PAN-Biotech | ST-30-3302 |
| Penicillin-Streptomycin | Hyclone Laboratories, Inc | J190007 |
| DMSO | Sigma-Aldrich Corporation | D8418 |
| A549 cells | Cobioer Biosciences Co., LTD | CBP60084 |
| Prostaglandin E2 Kit | PerkinElmer Corporation | 62P2APEG |
| 24-well plate | Corning United States Corporation | 3337 |
| 384-well plate | Corning United States Corporation | 3570 |
| Multifunctional microplate reader | BMG LABTECH Corporation | PHERAstar ® FSX |
| CO₂ cell incubator | Thermo Fisher Scientific | RI-250 |
| | Corporation | |
| microscope | Thermo Fisher Scientific | DMI1 |
| | Corporation | |

### 2. Experimental Method:

a) A549 cells were inoculated in the 24-well plate, and after cell adhesion, IL-1β was added thereto for 16 h of stimulation to induce COX2 expression and PGE2 production.
b) A solution of the compound to be tested was prepared with the culture medium and gradiently diluted to 3 concentrations of 10 nM, 300 nM and 10,000 nM or 7 concentrations of 0.64 nM, 3.2 nM, 16 nM, 80 nM, 400 nM, 2000 nM and 10,000 nM, and meanwhile the positive control group (only IL-1β was added to the cells for induction) and negative control group (only cells were added in the wells without any treatment) were set up. The cell supernatants were collected after 8 h of action, in which the positive control group was induced by IL-1β without treatment of the compounds, and the negative control group was neither stimulated by IL-1β, nor treated with the compounds.
c) The PGE2 content of the samples was determined by Prostaglandin E2 Kit, and the fluorescence signal was detected by a multifunctional microplate reader (Ex/Em=337/620, 337/665).

### 3. Data Analysis:

a) A standard curve was plotted with the PGE2 standard in the Prostaglandin E2 Kit, and the PGE2 concentration was calculated by substituting with the fluorescence signal of the sample.
b) The PGE2 upregulation rate % was calculated by using the following formula:
   PGE2 upregulation rate %=(PGE2 concentration of sample group/PGE2 concentration of positive control group)×100%.

### 4. Experimental Results

The PGE2 upregulation rates of the compounds of some Examples were showed in the table below,

| | PGE2 | PGE2 |
|---|---|---|
| | upregulation | upregulation |
| | rate at 10 nM | rate at 300 nM |
| | compound | compound |
| Example No. | concentration | concentration |
| Example 2 | 200% | 348% |
| Example 5 | 451% | / |
| Example 6 | 412% | 450% |
| Example 7 | 479% | 758% |
| Example 10 | / | 206% |
| Example 11 | 990% | 1380% |
| Example 12 | / | 170% |
| Example 13 | / | 207% |
| Example 15 | / | 168% |
| Example 21 | / | 211% |
| Example 22 | 282% | 329% |
| Example 23 | 254% | 375% |
| Example 24 | / | 310% |
| Example 25 | / | 276% |
| Example 26 | 499% | 2228% |
| Example 28 | / | 318% |
| Example 30 | / | 341% |
| Example 32 | 530% | 1324% |
| Example 33 | 393% | 754% |
| Example 35 | 208% | 231% |
| Example 36 | 301% | 384% |
| Example 40 | 233% | 315% |
| Example 42 | / | 204% |
| Example 45 | 177% | 347% |
| Example 46 | / | 251% |
| Example 47 | 424% | 774% |
| Example 63 | / | 222% |
| Example 64 | 173% | 196% |

In the table, “/” represents not detected. According to the above table, it can be seen that the compounds of the present application are able to achieve a PGE2 upregulation rate >100% in A549 cells, certain compounds of the present application are able to achieve a PGE2 upregulation rate >200% in A549 cells, certain preferred compounds of the present application are able to achieve a PGE2 upregulation rate >300% or higher in A549 cells, and thus the compounds of the present application may have a good intracellular PGE2 upregulation activity.

For the purpose of describing and disclosing, all patents, patent applications and other established publications are expressly incorporated herein by reference. These publications are provided solely for their disclosure prior to the filing date of this application. All statements regarding the dates of these documents or the representation of the contents of these documents are based on the information available to the applicants and do not constitute any admission as to the correctness of the dates of these documents or the contents of these documents. Moreover, any reference to these publications herein does not constitute an admission that the publications form part of the common general knowledge in the art in any country.

Those skilled in the art will recognize that the scope of the present application is not limited to the various specific embodiments and examples described above, but is capable of making various modifications, substitutions, or recombinations without departing from the spirit of the present application, and that these adjusted technical solutions fall within the protection scope of the present application.

1. A compound represented by formula (I), a stereoisomer, a tautomer or a mixed form thereof, or a pharmaceutically acceptable salt thereof, or a solvate thereof, or a prodrug thereof: 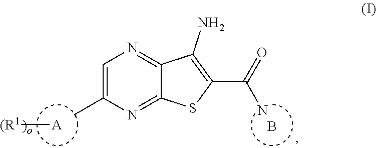
ring A is an aromatic ring, an aromatic heterocycle, or an unsaturated aliphatic heterocycle;
ring B is a 5-12 membered saturated aliphatic heterocycle or a ring formed by a 5-12 membered saturated aliphatic heterocycle fused with a benzene ring;
o is 0, 1, 2, or 3;
R¹ is each independentlydeuterium, tritium, nitro, hydroxyl, mercapto, halogen, cyano, ═O, imino, an amine group, an ester group, an aldehyde group, carboxyl, amido, C₃-C₈ cycloalkyl, C₁-C₆ alkyl, C₂-C₆ alkenyl, C₁-C₆ alkoxy, or a 3-8 membered saturated aliphatic heterocycle;
or when o is 2, or 3, any two R¹ together with atoms of ring A to which they are connected form a 3-8 membered alicyclic group, a 3-8 membered aliphatic heterocyclic group;
wherein the aromatic heterocycle, the saturated aliphatic heterocycle, the unsaturated aliphatic heterocycle, and the aliphatic heterocyclic group each independently comprise 1-3 heteroatoms independently the group consisting of N, O, and S, and the ring B comprises at least 1 nitrogen atom;
the ring B, and R¹ are optionally substituted by one or more independently the group consisting of deuterium, tritium, nitro, hydroxyl, an aldehyde group, an amine group, imino, halogen, cyano, an ester group, carboxyl, amido, —O, C₁-C₆ alkyl, C₁-C₆ alkoxy, C₃-C₈ cycloalkyl, 6-10 membered aryl, and 5-10 membered heteroaryl.

2. The compound, the stereoisomer, the tautomer or the mixed form thereof, or the pharmaceutically acceptable salt thereof, or the solvate thereof, or the prodrug thereof according to claim 1, wherein R¹ is each independently deuterium, tritium, nitro, hydroxyl, mercapto, halogen, cyano, ═O, imino, an amine group, an ester group, an aldehyde group, carboxyl, amido, C₃-C₈ cycloalkyl, C₁-C₆ alkyl, C₁-C₆ alkoxy, or a 3-8 membered saturated aliphatic heterocycle;
or when o is 2, or 3, any two R¹ together with atoms of ring A to which they are connected form a 3-8 membered alicyclic group, a 3-8 membered aliphatic heterocyclic group;
the R¹ is optionally substituted by one or more independently the group consisting of deuterium, tritium, nitro, hydroxyl, an aldehyde group, an amine group, imino, halogen, cyano, an ester group, carboxyl, amido, ═O, C₁-C₆ alkyl, C₁-C₆ alkoxy, C₃-C₈ cycloalkyl, 6-10 membered aryl, and 5-10 membered heteroaryl.

3. The compound, the stereoisomer, the tautomer or the mixed form thereof, or the pharmaceutically acceptable salt thereof, or the solvate thereof, or the prodrug thereof according to claim 1, wherein
ring B is 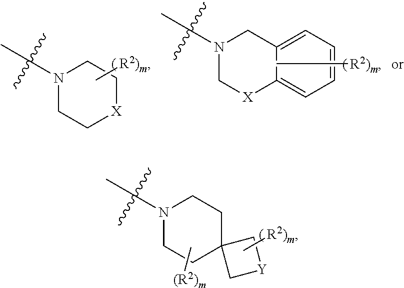
wherein X is a covalent bond, O, S, NH, (CH₂)ₙ, or SO₂; Y is a covalent bond, S, NH, (CH₂)ₙ, or SO₂; m is 0, 1, 2, or 3; R² is each independently deuterium, tritium, nitro, hydroxyl, mercapto, cyano, halogen, an amine group, an ester group, an aldehyde group, carboxyl, amido, C₁-C₆ alkyl, C₁-C₆ haloalkyl, C₁-C₆ alkoxy, C₃-C₈ cycloalkyl, 6-10 membered aryl, or 5-10 membered heteroaryl; n is 1, 2, or 3;

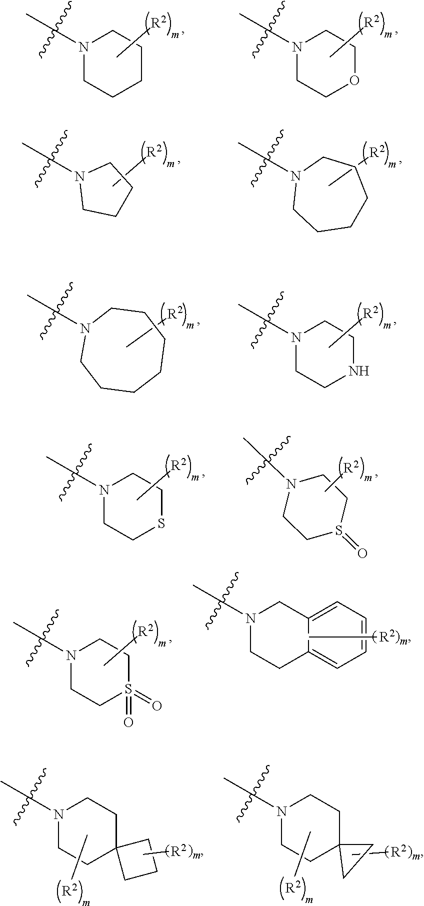

4. The compound, the stereoisomer, the tautomer or the mixed form thereof, or the pharmaceutically acceptable salt thereof, or the solvate thereof, or the prodrug thereof according to claim 3, wherein the R² is each independently deuterium, tritium, nitro, hydroxyl, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, or pyridyl.

5. The compound, the stereoisomer, the tautomer or the mixed form thereof, or the pharmaceutically acceptable salt thereof, or the solvate thereof, or the prodrug thereof according to claim 1, wherein the ring A is a 6-10 membered aromatic ring, a 5-10 membered aromatic heterocycle, or a 6-8 membered unsaturated aliphatic heterocycle.

6. The compound, the stereoisomer, the tautomer or the mixed form thereof, or the pharmaceutically acceptable salt thereof, or the solvate thereof, or the prodrug thereof according to claim 1, wherein
ring A is a 6-10 membered aromatic ring, a 5-10 membered aromatic heterocycle, or a 5-7 membered unsaturated aliphatic heterocycle;
ring B is a 5-9 membered saturated aliphatic heterocycle or a ring formed by a 5-7 membered saturated aliphatic heterocycle fused with a benzene ring;
o is 0, 1, or 2;
R¹ is each independently deuterium, tritium, hydroxyl, halogen, cyano, ═O, imino, an amine group, amido, C₅-C₇ cycloalkyl, C₁-C₆ alkyl, C₂-C₆ alkenyl, C₁-C₆ alkoxy, or a 5-7 membered saturated aliphatic heterocycle; or, any two R¹ together with atoms of ring A to which they are connected form a 5-6 membered alicyclic group, a 5-6 membered aliphatic heterocyclic group;
wherein the aromatic heterocycle, the saturated aliphatic heterocycle, the unsaturated aliphatic heterocycle, and the aliphatic heterocyclic group each independently comprise 1-3 heteroatoms independently the group consisting of N, O, and S, and ring B comprises at least 1 nitrogen atom;
the ring B is a monocycle, a fused ring or a spirocycle, and is optionally substituted by one or more independently the group consisting of deuterium, tritium, hydroxyl, halogen, ═O, C₁-C₆ alkyl, C₁-C₆ alkoxy, and C₃-C₅ cycloalkyl;
the R¹ is optionally substituted by one or more independently the group consisting of deuterium, tritium, C₁-C₆ alkyl, C₁-C₆ alkoxy, and C₃-C₆ cycloalkyl.

7. The compound, the stereoisomer, the tautomer or the mixed form thereof, or the pharmaceutically acceptable salt thereof, or the solvate thereof, or the prodrug thereof according to any one of claim 1, wherein the compound has a structure represented by formula II, 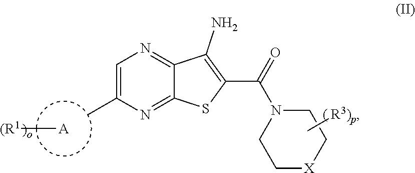
wherein X is a covalent bond, S, CH₂, (CH₂)₂ or (CH₂) 3; R³ is each independently deuterium, tritium, nitro, hydroxyl, mercapto, cyano, halogen, an amine group, an ester group, an aldehyde group, carboxyl, amido, C₁-C₆ alkyl, C₁-C₆ haloalkyl, C₁-C₆ alkoxy, C₃-C₈ cycloalkyl, 6-10 membered aryl, or 5-10 membered heteroaryl; p is 0, or 1.

8. The compound, the stereoisomer, the tautomer or the mixed form thereof,
or the pharmaceutically acceptable salt thereof, or the solvate thereof, or the prodrug thereof according to claim 7, wherein p is 0.

9. The compound, the stereoisomer, the tautomer or the mixed form thereof, or the pharmaceutically acceptable salt thereof, or the solvate thereof, or the prodrug thereof according to claim 8, wherein p is 0 and X is CH₂.

10. The compound, the stereoisomer, the tautomer or the mixed form thereof, or the pharmaceutically acceptable salt thereof, or the solvate thereof, or the prodrug thereof according to claim 7, wherein ring A is a 6-10 membered aromatic ring, a 5-10 membered aromatic heterocycle, or a 6-8 membered unsaturated aliphatic heterocycle

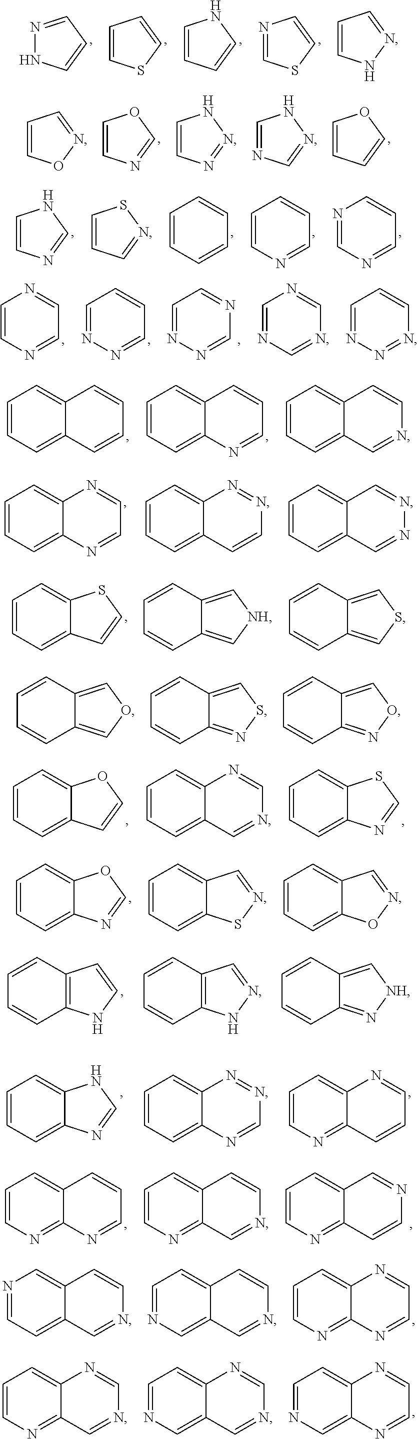

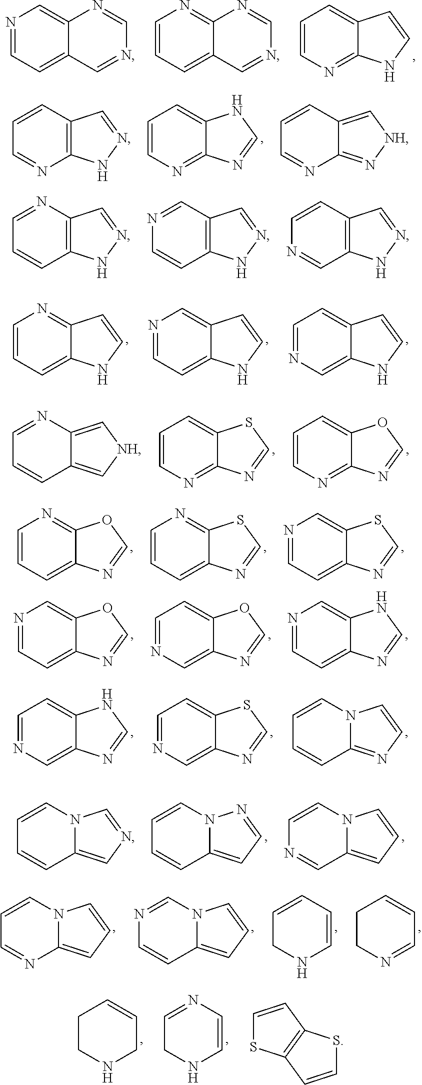

11. The compound, the stereoisomer, the tautomer or the mixed form thereof, or the pharmaceutically acceptable salt thereof, or the solvate thereof, or the prodrug thereof according to claim 1, wherein R¹ is each independently deuterium, tritium, nitro, hydroxyl, mercapto, halogen, cyano, —O, imino, an amine group, an ester group, an aldehyde group, carboxyl, amido, cyclopropyl, cyclobutyl, cyclohexyl, cyclopentyl, methyl, ethyl, isopropyl, n-butyl, isobutyl, t-butyl, n-pentyl, iso-pentyl, tert-amyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, n-butoxy, isobutoxy, t-butoxy, n-pentoxy, isopentoxy, tert-pentyloxy, or n-hexyloxy; or any two R¹ together with atoms of ring A to which they are connected form dioxanyl, dioxolanyl, dioxenyl, dioxolyl, dihydropyridyl, 3-pyrrolinyl, wherein the R¹ is optionally substituted by one or more independently the group consisting of deuterium, tritium, nitro, hydroxyl, —NH₂, mercapto, halogen, cyano, an ester group, carboxyl, amido, —O, ═NH, C₁-C₆ alkyl, C₁-C₆ alkoxy, C₃-C₈ cycloalkyl, 6-10 membered aryl, and 5-10 membered heteroaryl.

12. The compound, the stereoisomer, the tautomer or the mixed form thereof, or the pharmaceutically acceptable salt thereof, or the solvate thereof, or the prodrug thereof according to claim 1, wherein R¹ is each independently deuterium, tritium, nitro, hydroxyl, mercapto, halogen, cyano, ═O, imino, an amine group, an ester group, an aldehyde group, carboxyl, amido, cyclopropyl, cyclobutyl, cyclohexyl, cyclopentyl, methyl, ethyl, isopropyl, n-butyl, isobutyl, t-butyl, n-pentyl, iso-pentyl, tert-amyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, n-butoxy, isobutoxy, t-butoxy, n-pentoxy, isopentoxy, tert-pentyloxy, or n-hexyloxy; or any two R¹ together with atoms of ring A to which they are connected form dioxanyl, dioxolanyl, wherein the R¹ is optionally substituted by one or more independently the group consisting of deuterium, tritium, nitro, hydroxyl, —NH₂, mercapto, halogen, cyano, an ester group, carboxyl, amido, ═O, ═NH, C₁-C₆ alkyl, C₁-C₆ alkoxy, C₃-C₈ cycloalkyl, 6-10 membered aryl, and 5-10 membered heteroaryl.

13. The compound, the stereoisomer, the tautomer or the mixed form thereof, or the pharmaceutically acceptable salt thereof, or the solvate thereof, or the prodrug thereof according to claim 1, wherein the compound is:

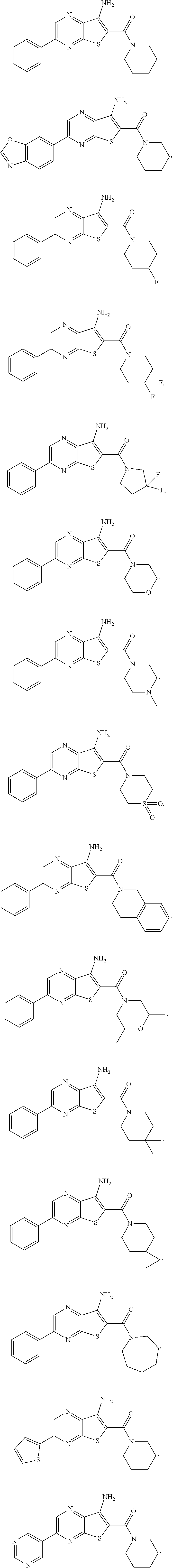

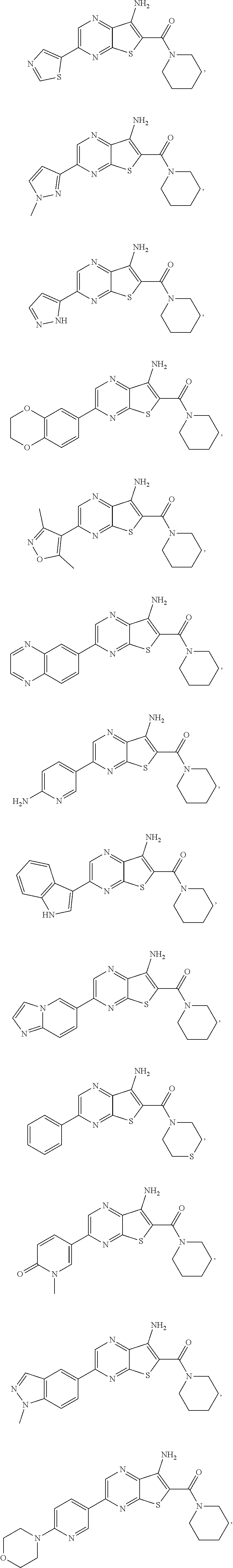

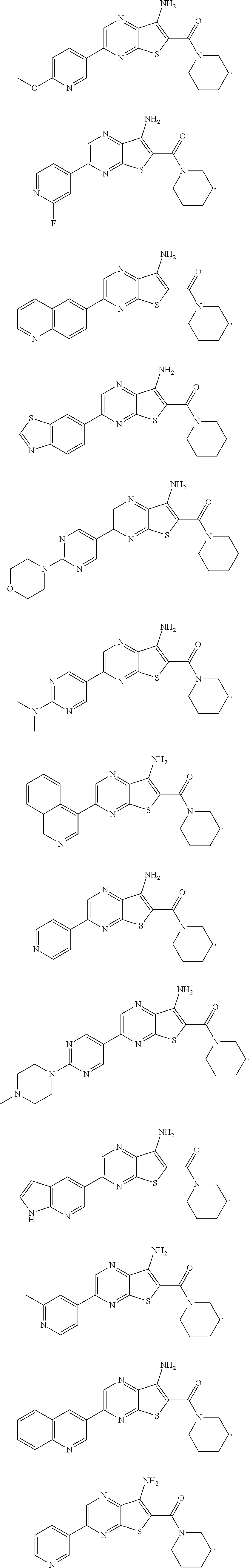

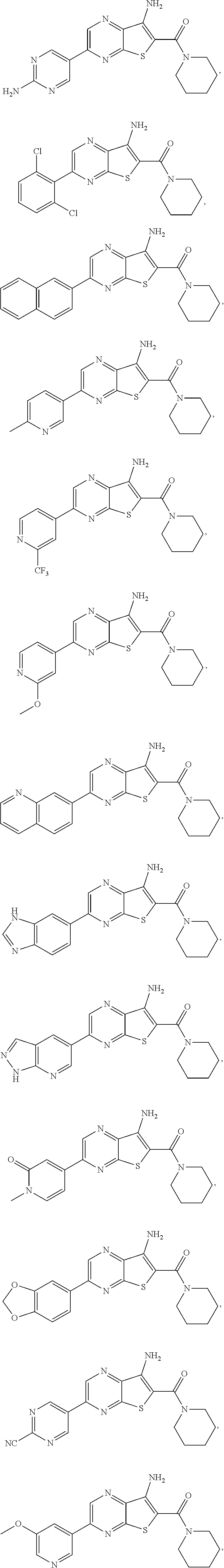

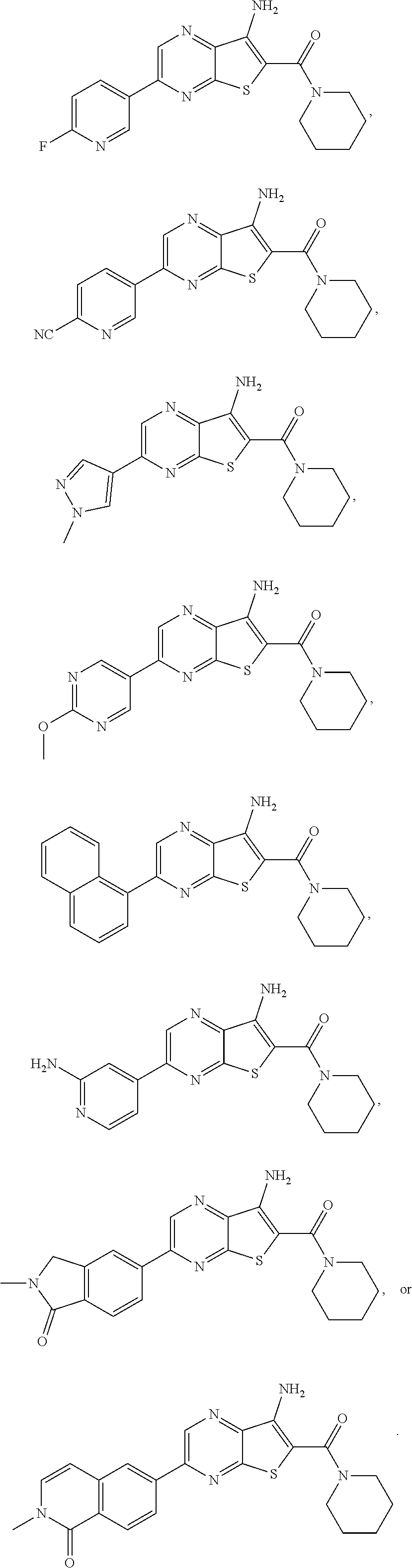

14. A pharmaceutical composition, comprising at least one of the compound, the stereoisomer, the tautomer or the mixed form thereof, or the pharmaceutically acceptable salt thereof, or the solvate thereof, or the prodrug thereof according to claim 1, and at least one pharmaceutically acceptable excipient.

15. (canceled)

16. The compound, the stereoisomer, the tautomer or the mixed form thereof, or the pharmaceutically acceptable salt thereof, or the solvate thereof, or the prodrug thereof according to claim 3, wherein the ring B is

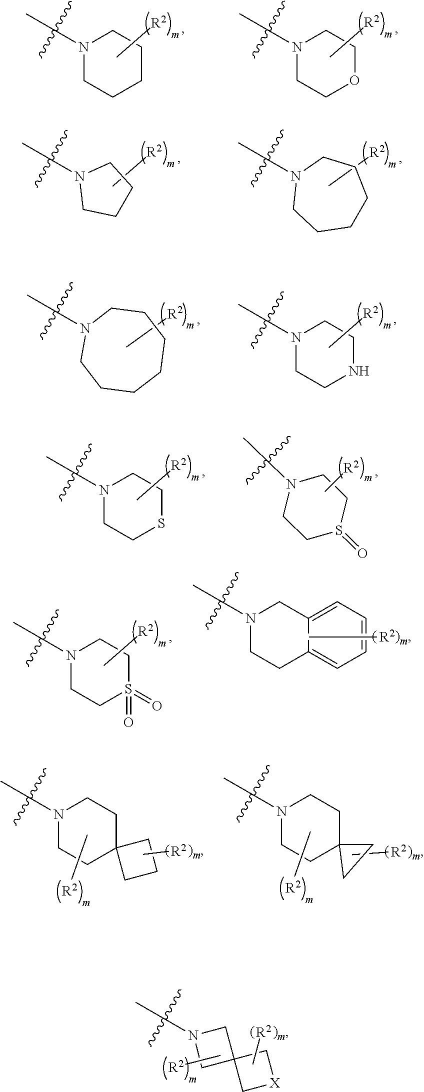

17. The compound, the stereoisomer, the tautomer or the mixed form thereof, or the pharmaceutically acceptable salt thereof, or the solvate thereof, or the prodrug thereof according to claim 5, wherein the aromatic ring, and the aromatic heterocycle are a monocycle or a fused ring, the unsaturated aliphatic heterocycle is a monocycle, and the aromatic heterocycle and the unsaturated aliphatic heterocycle each independently comprise 1-3 heteroatoms independently the group consisting of N, O, and S.

18. The compound, the stereoisomer, the tautomer or the mixed form thereof, or the pharmaceutically acceptable salt thereof, or the solvate thereof, or the prodrug thereof according to claim 7, wherein the R³ is each independently deuterium, tritium, nitro, hydroxyl, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, or pyridyl.

19. The compound, the stereoisomer, the tautomer or the mixed form thereof, or the pharmaceutically acceptable salt thereof, or the solvate thereof, or the prodrug thereof according to claim 5, wherein the ring A is

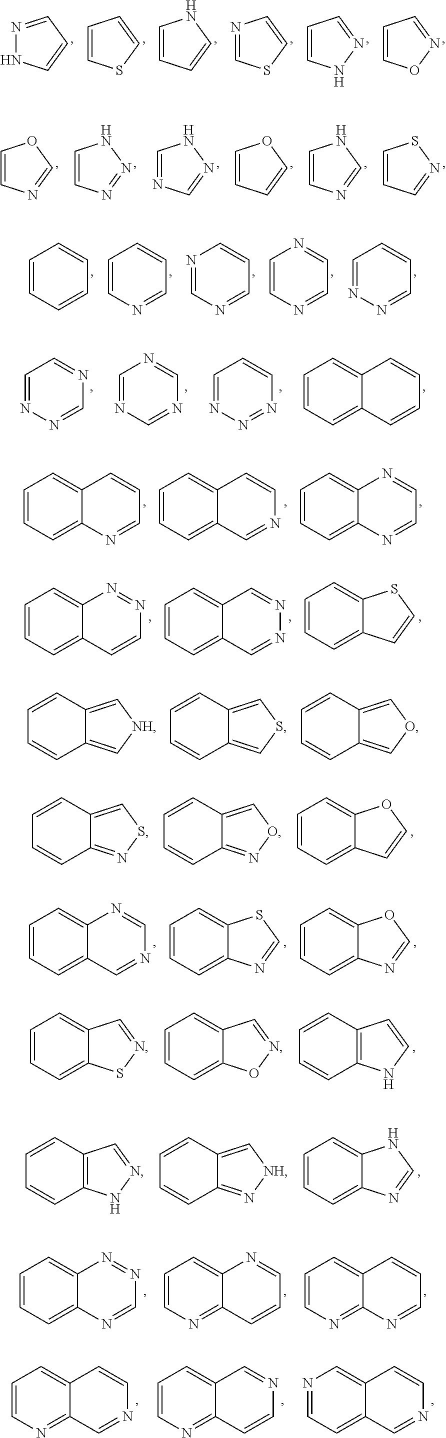

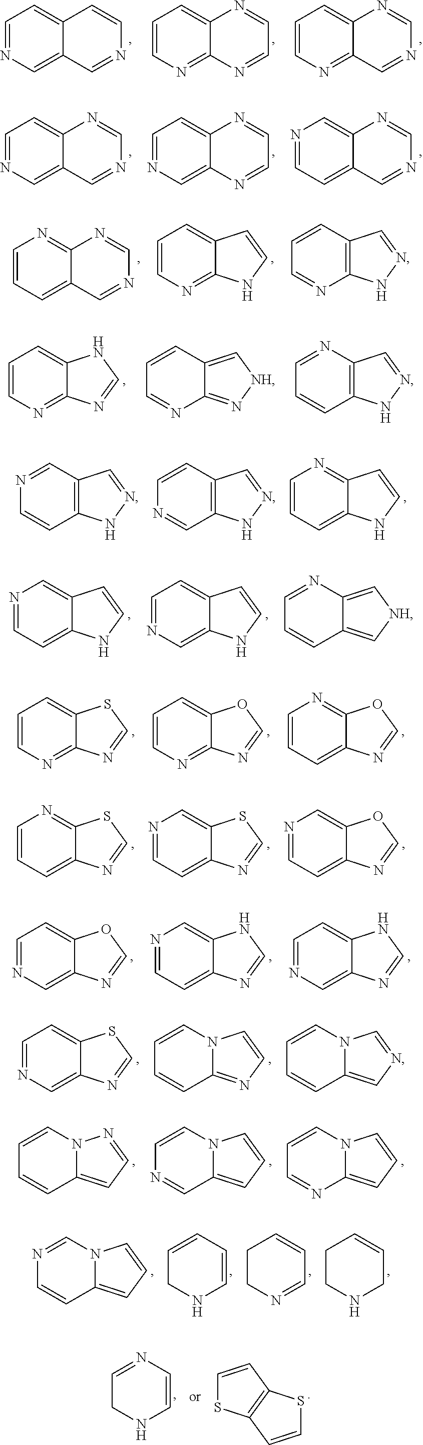

20. A method of treating or preventing a 15-PGDH associated disease, comprising administering to a subject in need thereof a compound, a stereoisomer, a tautomer or a mixed form thereof, or a pharmaceutically acceptable salt thereof, or a solvate thereof, or a prodrug thereof according to claim 1, or a pharmaceutical composition comprising the same.

21. The method according to claim 20, wherein the method is used for treating or preventing fibrosis, oral ulcer, gum disease, colitis, ulcerative colitis, gastroduodenal ulcer, inflammatory disease, vascular insufficiency, Raynaud's disease, Buerger's disease, neuropathy, pulmonary arterial hypertension, cardiovascular and renal disease, cardiovascular disease, trauma, skin damage, autoimmune disease, graft-versus-host disease, osteoporosis, ear disease, eye disease, neutropenia, diabetes mellitus, underactive bladder, or for promoting hair growth, pigmentation, tissue repair, tissue regeneration, promotion of implant in stem cell transplantation or bone marrow transplantation or organ transplantation, neurogenesis and neuronal cell death, or muscle regeneration and cervical ripening, or for enhancing resistance to the toxicity of radiation exposure, the toxicity of chemotherapy, the toxicity of immunosuppressant.
